(19) United States
(12) Patent Application Publication
(10) Pub. No.: US 2009/0011980 A1
(43) Pub. Date: Jan. 8, 2009

(54) Method of Treating Parkinson's Disease in Humans by Direct Infusion of Glial Cell-Line Derived Neurotrophic Factor Into the Zona Incerta

(76) Inventor: Steven Streatfield Gill, Bristol (GB)

Correspondence Address: KOHN & ASSOCIATES, PLLC, 30500 NORTHWESTERN HWY, SUITE 410, FARMINGTON HILLS, MI, 48334, US

(21) Appl. No.: 11/629,254

(22) PCT Filed: Jun. 13, 2005
(86) PCT No.: PCT/GB05/02332

§ 371 Date: Sep. 17, 2008

(30) Foreign Application Priority Data
Jun. 11, 2004 (GB) 0413077.9

(51) Int. Cl. A61K 38/18; A61P 25/00

(52) U.S. Cl. 514/12

(57) ABSTRACT

A method of treating Parkinson's disease in humans is disclosed, wherein glial cell-line derive neurotrophic factor (GDNF) is chronically administered directly to the SNc and at least one subthalamic region or, alternatively, to the SNc, at least one sub thalamic region, and at least one putaminal region of the brain of a human in need of treatment thereof. In one aspect of the present invention the GDNF is infused directly into at least one caudal zona incerta and at least one postero-dorsal putamen of the brain through indwelling intraparenchymal brain catheters connected to an implantable pump.

The present invention relates generally to the field of neurobiology. More particularly, it concerns methods for treating Parkinson's disease in humans and related methods of restoring atrophic dopaminergic neurons and protecting dopaminergic neurons at risk of degeneration are also described.

Idiopathic Parkinson's disease (PD) is a neurodegenerative disorder characterized by the progressive death of selected populations of dopaminergic neurons, particularly within the pars compacta of the substantia nigra (SNc), with resulting reduction in striatal dopamine levels. There are approximately 500,000 specialized dopaminergic cells in the pars compacta of the substantia nigra of young adults. Symptoms of parkinsonism emerge when 75-80% of the dopaminergic innervation is destroyed. The consequential cardinal features, upon which clinical diagnosis is based, are tremor, rigidity, postural instability and akinesia/bradykinesia (Lang and Lozano, 1998). There are reportedly more than one million affected individuals in North America (Lang and Lozano, 1998), and an estimated overall prevalence within Europe of 1.6 per 100 population aged 65 years or older (de Rijk et al., 1997). Mortality among affected individuals is 2 to 5 times greater than among their age-matched, unaffected peers (Bennett et al., 1996; Morens et al., 1996; Louis et al., 1997), and life expectancy is markedly reduced (Morens et al., 1996). The single most consistent risk factor for the disease is age, and given the changing demography of industrialized nations, its burden upon their societies is likely to increase. Orally administered L-dopa, the immediate precursor of dopamine that is absorbed through the small intestine and is able, unlike dopamine itself, to cross the blood-brain barrier, remains the most effective treatment when combined with an aromatic amino acid decarboxylase inhibitor, currently widely available for Parkinson's disease (Koller, 2000; Jankovic, 2002). Although L-dopa does relieve the symptoms of PD (indeed, responsiveness to it, exhibited by more than 90% of patients, is one of the characteristic features of the disease (Lang and Lozano, 1998), its use is not without problems. Its principal limitation, shared by dopamine agonists and more clearly apparent after several years of treatment, is the increasing inconsistency of patient responsiveness, manifested by motor fluctuations that take the form of distinct “wearing-off” and “on-off” phenomena (Nutt and Holford, 1996: Lang and Lozano, 1998; Koller, 2000; Jankovic, 2002). “Wearing-off,” also described as “end of dose deterioration,” is the term given to the relatively gradual and predictable decline in response to a dose of L-dopa that occurs over time, and this contrasts with “on-off” fluctuations in motor performance that are not clearly related to L-dopa dosing. In their early stages, motor fluctuations may be mitigated by approaches that prolong the actions of L-dopa (e.g., slow release formulations of the molecule or the co-administration of a catechol-O-methyl-transferase inhibitor) or by the use of longer-acting synthetic dopamine agonists; however, these interventions cannot prevent an eventual increased unpredictability and lessened control of motor fluctuations and an increased incidence of dyskinesias during “on” periods (Lang and Lozano, 1998; Koller, 2000; Jankovic, 2002).

Glial cell line-derived neurotrophic factor (GDNF) is a distant member of the transforming growth factor beta superfamily and was first isolated from the culture medium of a rat glial cell line as a potent neurotrophic factor described as having relative specificity for dopaminergic neurons within dissociated rat embryonic midbrain cultures (Lin et al., 1993; Lin et al., 1994). It has since been shown to exert an effect on other neuronal populations as well. GDNF is important for the development and maintenance of dopaminergic, serotoninergic, noradrerergic and glutametergic neurons (Lin et al., 1993; Lin et al., 1994; Arenas et al., 1995; Beck et al., 1996; Martin et al., 1996). The human GDNF gene has been cloned, and recombinant human GDNF displaying full biologic activity has been expressed in E. coli (Lin et al., 1993).

Data collected in cell culture (Lin et al., 1993; Lin et al., 1994; Hou et al., 1996) and in rodent models of PD showed GDNF to be neuroprotective, encourage fibre outgrowth and improve motor function when delivered into the cerebral ventricles or directly into the striatum or substantia nigra (Hoffer et al., 1994; Bowenkamp et al., 1995; Tomac et al., 1995a; Kearns et al., 1995). Intraparenchymal delivery in animal models has been shown to be effective whether delivered by bolus injection, chronic infusion using a pump, or by infecting the brain with live replication deficient viral particles engineered to deliver GDNF (Gash, et al., 1998; Grondin, et al., 2002b; Kordower, et al., 2000). Therefore, GDNF has long held significant therapeutic promise for the treatment of human neurodegenerative disorders such as Parkinson's disease (PD) and amyotrophic lateral sclerosis (ALS) (Gash et al., 1996). However, important obstacles against the therapeutic application of GDNF to PD and other human neurological disorders have been encountered. First, GDNF is a macromolecule that cannot pass through the blood-brain barrier, making it difficult to therapeutically deliver GDNF to the human brain. Secondly, animal models have proved to be limited in their relevance to the human condition because of significant differences in the relative size of the brain. Intraventricular infusion of GDNF has, in fact, been attempted in PD and ALS patients but failed to result in therapeutic benefits. More specifically, in four clinical studies in subjects with idiopathic PD (53 subjects; 50 of these were enrolled in the double-blind, placebo-controlled trial, and 38 of 50 received study drug), GDNF delivered to the cerebral ventricles (ICV) by monthly bolus dose (25 to 4000 μg per dose) or by chronic infusion (3 to 50 μg/day) failed to demonstrate clinical efficacy, i.e., no clinically or statistically significant improvements in signs or symptoms of PD were observed (Nutt, et al., 2003). In addition, almost all subjects (92% to 100%) experienced at least one adverse event during the studies. Mild-to-moderate nausea was the most frequently reported adverse event (approximately 70% to 90% incidence across all studies). Mild-to-moderate paresthesia was reported in 30% to 80% of subjects across all studies. Weight loss was reported in 14% to 63% of subjects across all studies. Serious adverse events were reported in 21% to 44% of subjects across all studies. Furthermore, there was no evidence of restoration of dopamine fibers in the striatum in one subject at post-mortem (Kordower, et al., 1999).

Another seemingly promising approach to treating PD that failed in clinical trials was the implantation of embryonic dopaminergic neurons into the brains of patients with PD. In a randomized, double-blind trial in which patients either received intraputaminal transplants of cultured embryonic mesencephalic tissue or were given sham surgery in which the dura mater was not penetrated, no clinical improvement was observed as a result of the transplants in patients over 60 years of age at one year after surgery, and only moderate improvement was apparent in those aged 60 years or less (Freed, C. et al., 2001). During continued follow-up of 12 to 36 months in patients who had received transplants, dystonia and dyskinesias had developed in a number of patients, all of whom had been <60 years of age at the time of surgery and each of whom had experienced clinical improvement during the first year after transplantation. Investigators in this study later reported findings that suggest that unbalanced increases in dopaminergic function resulted in the undesirable outcomes of neuronal transplantation for parkinsonism (Ma, Y. et al., 2002).

Until recently no effective method existed to prevent or repair the damage caused by neuropathies, such as Parkinson's disease (parkinsonism) in human patients. Recently, however, Gill et al. demonstrated that GDNF delivered directly into the postero-dorsal putamen of five patients with idiopathic PD by chronic infusion using a pump and catheter system was safe and well tolerated (Gill, et al., 2003). All aspects of the disease improved with chronic intraputamenal GDNF infusion except tremor and dystonia scores (Part III and IV of the Unified Parkinson's Disease rating score). Furthermore, positron emission tomography (PET) scans showed increased ¹⁸F-dopa uptake in the immediate vicinity of the catheters by as much as 80%. Despite the significant positive outcomes seen in these studies, the methods of treatment used in these studies failed to significantly alleviate tremor, a hallmark symptom of PD. In fact, two patients who had tremor predominant disease at the beginning of the study noticed a gradual worsening of tremor and dystonia. Both of these patients also experienced dystonic posturing of their feet when the effect of L-dopa medications were “wearing off”. As symptoms of tremor substantially affect the quality of life of PD patients and do not respond to medications such as high dose anti-cholinergics, there exists a need for methods of treating PD which alleviate symptoms of tremor as well as the other debilitating effects of PD.

Accordingly, it is an object of the present invention to provide methods of treating PD in humans comprising the chronic infusion of GDNF into specific regions of the human brain which allow for the alleviation of a broader spectrum of PD-associated symptoms including tremor. This and other such objectives will be readily apparent to the skilled artisan from this disclosure.

The present invention is based on the premise that the progression of symptoms of tremor and dystonia in PD patients who have received intraputaminal GDNF infusion is due to the continued loss of dopamine to the subthalamic regions and the less than optimal up regulation of dopamine delivery to the basal ganglia of the afflicted brain. Therefore, continuous delivery of GDNF to the SNc or a subthalamic region of the brain, such as the subthalamic nucleus, or zona incerta, of a PD patient or, alternatively, at least one putaminal region and to the SNc, or a subthalamic region of the brain of a PD patient by means of an implantable pump and one or more indwelling catheters will lead to dramatic anti-parkinsonian and anti-dyskinetic effects, including tremor. Because degeneration of both midbrain dopaminergic and non-dopaminergic (serotoninergic, cholinergic, and noradrenergic) occurs in PD, the methods of the present invention may be further associated with re-innervation and/or restoration of neurotransmitter stores in previously neurotransmitter deficient neurons in human patients afflicted with PD.

A first aspect of the present invention concerns a method of treating Parkinson's disease in a human comprising administering a pharmaceutical composition comprising a pharmaceutically effective dose of a GDNF protein product to the SNc or a subthalamic region of the brain of a PD patient. Another aspect of the present invention concerns a method of treating Parkinson's disease in a human comprising administering a pharmaceutical composition comprising a pharmaceutically effective dose of a GDNF protein product to at least one putaminal region and to at least one of the SNc and a subthalamic region of the brain of a PD patient. The GDNF protein product includes, without limitation, a pharmaceutically effective dose of r-metHuGDNF (a dimeric protein having an the amino acid sequence shown below in Table 1) or variants and/or derivatives thereof.

Applicants also disclose herein the use of a pharmaceutically effective amount of GDNF and at least one pharmaceutically acceptable vehicle, excipient, or diluent in the preparation of a pharmaceutical composition for treating Parkinson's disease, wherein the composition is for administration to the SNc or a subthalamic region such as the subthalamic nucleus and the zona incerta or, alternatively, to at least one putaminal region and to at least one of the SNc and a subthalamic region, such as the subthalamic nucleus and the zona incerta of the brain of a human patient in need thereof.

The methods of the present invention are contemplated as being useful in repairing neural pathways damaged by Parkinson's disease in humans. In addition, the methods of the present invention may restore widespread neural cell function in a patient having Parkinson's disease to levels not possible by current PD therapies. Specifically, the methods described herein are capable of stimulating nerve regeneration, including re-innervation of damaged human brain tissue by neurons including, but not limited to, dopaminergic neurons. In a preferred embodiment there is provided a method of increasing the function of dopaminergic neurons that comprises administering a pharmaceutically effective dose of r-metHuGDNF to at least one of the SNc and at least one subthalamic region such as the subthalamic nucleus and the zona incerta or, alternatively, to at least one putaminal region and a least one of the SNc and a subthalamic region of the brain of a human patient in need thereof.

The present invention also concerns the use of a pharmaceutically effective amount of GDNF and at least one pharmaceutically acceptable vehicle, excipient, or diluent in the preparation of a pharmaceutical composition for increasing the function of neurons, wherein the composition is for administration to at least one of the SNc and subthalamic region such as the subthalamic nucleus and the zona incerta or, alternatively, to at least one putaminal region and at least one of the SNc and a subthalamic region such as the subthalamic nucleus and the zona incerta of the brain of a human patient in need thereof.

Additionally provided are methods of treating cognitive disorders in humans that comprise administering a pharmaceutically effective dose of r-metHuGDNF to at least one of the SNc and a subthalamic region such as the subthalamic nucleus and the zona incerta or, alternatively, to at least one putaminal region and at least one of the SNc and a subthalamic region, such as the subthalamic nucleus and the zona incerta of the brain of a human patient in need thereof. Preferred methods of treating cognitive disorders in humans comprise administering a pharmaceutically effective dose of r-metHuGDNF to at least one of the SNc and a subthalamic region such as the subthalamic nucleus or zona incerta or, alternatively, to at least one putaminal region and at least one of the SNc and a subthalamic region, such as the subthalamic nucleus and the zona incerta of the brain of a human patient in need thereof. A more preferred method of treating cognitive disorders in humans comprises administering a pharmaceutically effective dose of r-metHuGDNF to the lateral SNc and/or the caudal zona incerta. A most preferred method of treating cognitive disorders in humans comprises administering a pharmaceutically effective dose of r-metHuGDNF to the posterodorsal putamen and the lateral SNc and/or the caudal zona incerta of the brain of a human patient in need thereof.

In the methods and uses described herein, the preferred sites for administering the pharmaceutical composition comprising GDNF or pharmaceutically effective dose of GDNF includes the caudal zona incerta, in particular the area posterior to the subthalamic nucleus and about 2 mm above the SNc and the antral or postero-dorsal regions of one of both putamen.

In still other embodiments of the present invention methods of treating PD or cognitive disorders comprising the administration of a pharmaceutically effective dose of r-metHuGDNF to at least one of the SNc and a subthalamic region or, alternatively, to at least one putaminal region and at least one of the SNc and a subthalamic region of the brain of a human patient in need thereof further comprise assessing dopaminergic, noradrenergic and/or serotonergic function in the brain of said human pre-operatively, and, optionally, periodically post-operatively.

The methods of administering GDNF to at least one of the SNc and a subthalamic region or, alternatively, to at least one putaminal region and at least one of the SNc and a subthalamic region of the brain of a human patient in need thereof disclosed herein may also provide a prophylactic function in humans. Prophylactic administration may have the effect of preserving neural cell function in a human having, or at risk of having, PD. According to the invention, r-metHuGDNF administration to at least one of the SNc and a subthalamic region such as the subthalamic nucleus and the zona incerta or, alternatively, at least one putaminal region and at least one of the SNc and a subthalamic region such as the subthalamic nucleus and the zona incerta of the brain of a human patient in need thereof are contemplated to preserve the integrity of the nigrostriatal pathway in the human brain. Prophylactically administered r-metHuGDNF in accordance with the invention is also contemplated as a method of preventing or treating degeneration of the nigrostriatal pathway or loss of functional dopaminergic, serotonergic, and/or noradrenergicactivity associated with Parkinson's disease.

Additionally provided by the invention is a method for identifying a target area for the delivery of a pharmaceutically effective amount of GDNF for the treatment of Parkinson's disease, for increasing the function of dopaminergic neurons, for regenerating dopaminergic neurons or for protecting dopaminergic neurons susceptible to damage the method comprising using a scan of a patient's brain to identify one or more target areas selected from the SNc and a subthalamic region, or, alternatively, identifying one or more putaminal regions as a target area and at least one of the SNc and a subthalamic region as another target area. Preferably the subthalamic region is the STN or ZI, more preferably the caudal ZI, most preferably the caudal ZI posterior to the STN and approximately 2 mm above the SNc.

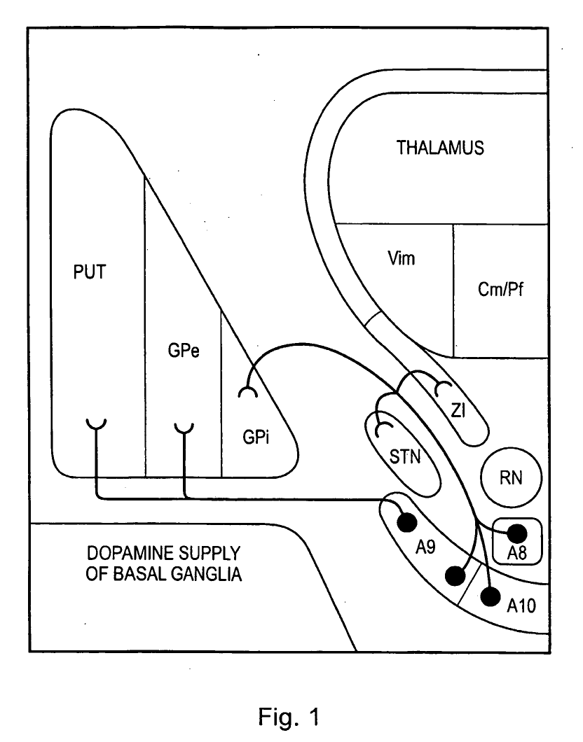

FIG. 1 illustrates the relevant anatomical structures of the brain dopamine supply of basal the ganglia and associated structures.

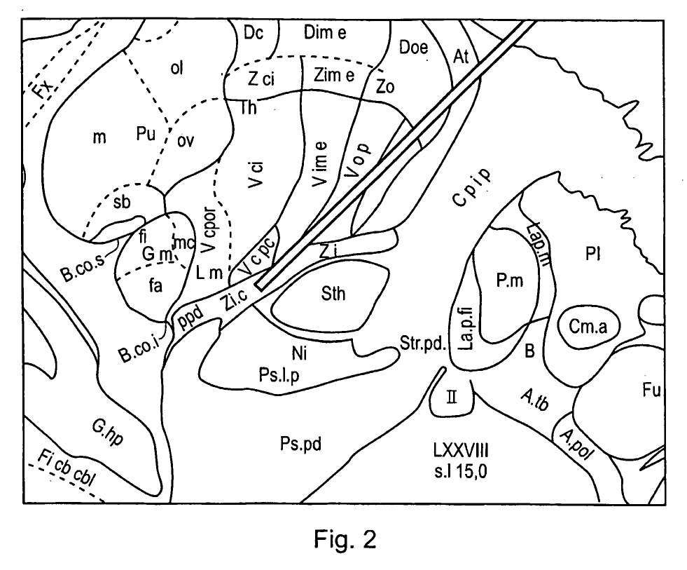

FIG. 2 illustrates a preferred target site for infusion of GDNF via a catheter. This saggital view image was taken from the Schaltenbrand Atlas and was modified to show the trajectory preferred placement of the catheter tip. The catheter tip is positioned as shown in the ZI.c, posterior to the STN and =2 mm above the lateral SN area A9 (Ni).

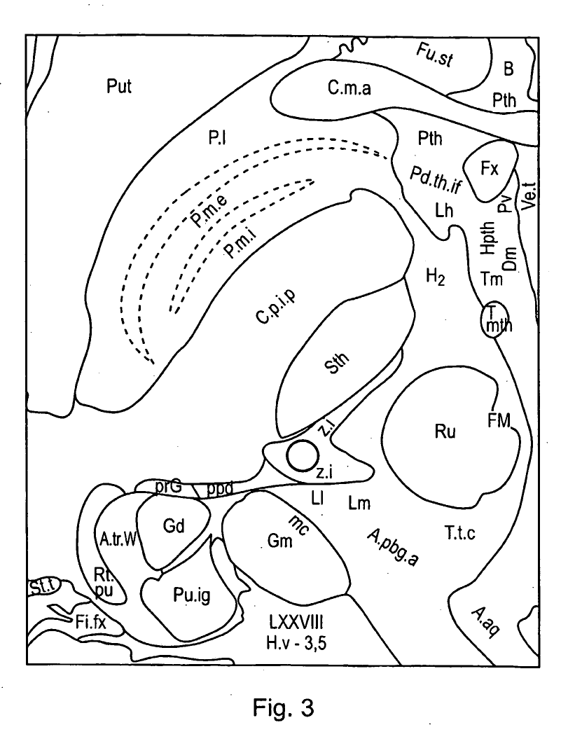

FIG. 3 illustrates a preferred target site for infusion of GDNF via a catheter. This axial view image was taken from the Schaltenbrand Atlas and was modified to show the preferred placement of the catheter tip. The catheter tip is positioned as in the ZI.c, posterior to the STN and 2 mm above the lateral SN area A9 (Ni). The catheter tip is shown by an asterisk (*).

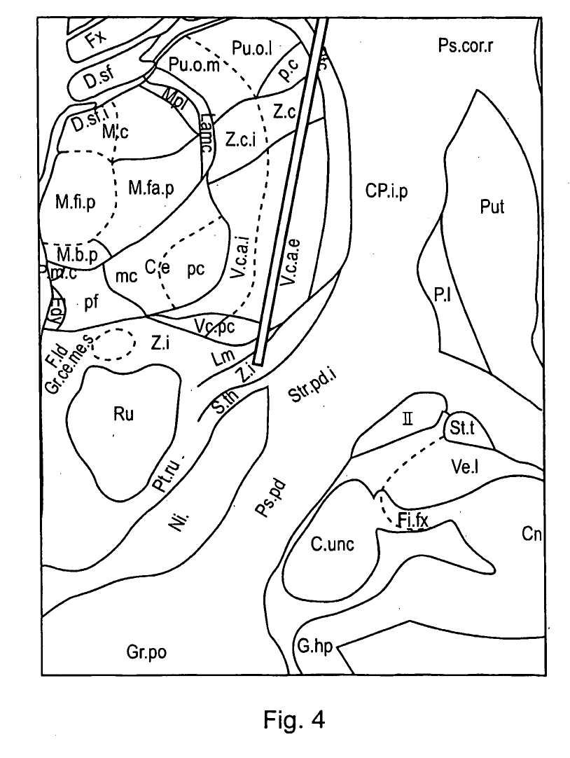

FIG. 4 illustrates a preferred target site for infusion of GDNF via a catheter. This coronal view image was taken from the Schaltenbrand Atlas and was modified to show the trajectory preferred placement of the catheter tip. The catheter tip is positioned as shown in the ZI.c, posterior to the STN and ≈2 mm above the lateral SN area A9 (Ni).

The section headings used herein are for organizational purposes only and are not to be construed as limiting the subject matter described.

### Abbreviations

In the preceding description and the experimental disclosure which follows, the following abbreviations apply:

6-OHDA 6-hydroxydopamine
ALS amyotrophic lateral sclerosis
ASA acute systemic anaphylaxis
AUC area under the concentration vs time curve

### CAPIT Core Assessment Program for Intracerebral Transplantations

CAPS 3-(cyclohexylamino)-1-propanesulfonic acid
CHO Chinese hamster ovary
CI continuous infusion
CM/Pf centromedian and parafascicular nuclei of the thalamus
CSF cerebrospinal fluid
CT computed tomography
DA dopamine, dopaminergic
DOPAC 3,4-dihydroxyphenylacetic acid
E. coli Escherichia coli

### FCA Freund's Complete Adjuvant

GDNF glial cell line-derived neurotrophic factor
Gpe globus pallidus externus
GPi globus pallidus internus

### GLP Good Laboratory Practice

HPLC high-performance liquid chromatography
HVA homovanillic acid
ICV intracerebroventricular
IM intramuscular
ISN intranigral
IT intrathecal
IV intravenous
L-dopa 3,4-dihydroxyphenylalanine (levodopa)
r-metHuGDNF recombinant-methionyl human GDNF
MPTP 1-methyl-4-phenyl-1,2,3,6-tetrahydropyridine
Ni or SNc lateral substantia nigra¹
PD Parkinson's disease
PET positron-emission tomography
pmn progressive motor neuropathy
Put putamen
Ret receptor tyrosine kinase
RN red nucleus
r-metHuGDNF recombinant-methionyl human GDNF
SC subcutaneous
SDS-PAGE sodium dodecylsulfate-polyacrylamide gel electrophoresis
SEM standard error of the mean
SN substantia nigra
SNc or Ni substantia nigra, pars compacta²
SNr substantia nigra, pars reticulata
STN subthalamic nucleus
TGF transforming growth factor
TH tyrosine hydroxylase
TH+ tyrosine hydroxylase positive

### UPDRS Unified Parkinson's Disease Rating Scale

ZI zona incerta
ZI.c caudal zona incerta

Each of the applications and patents and articles cited hereinafter, and each document cited or referenced in each of the applications and patents and articles cited hereinafter, including during the prosecution of each of the applications and patents cited hereinafter (“application and article cited documents”), and any manufacturer's instructions or catalogues for any products cited hereinafter or mentioned in each of the applications and patents and articles cited hereinafter and in any of the application and article cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

### DEFINITIONS

Unless otherwise noted, technical terms are used according to conventional usage. As utilized in accordance with the present disclosure, the following terms shall be understood to have the following meanings:

As used herein, the term “catheter” refers to any tubular medical device for insertion into a cavity, tissue, organ, or any substructure thereof of a living mammal to permit injection of a therapeutic agent. As particularly used here, a catheter is used to administer or deliver r-metHuGDNF to the brain or substructures thereof such as the ZI and/or the putamen. An “indwelling” catheter is one that is implanted and left in place for protracted periods, such as fifteen minutes or longer.

As used herein, the phrase “catheter system” refers to the combination of at least one catheter and at least one accessory device including, but not limited to, an anchor, stylet, guide tube, guide wire or a combination thereof.

“Continuous delivery” or “chronic infusion” are interchangeable and are intended to mean delivery of a substance over a period of time such that the procedure is distinguished from “bolus” delivery. Continuous delivery generally involves the delivery of a substance over a period of time without interruption. The rate of delivery need not be constant, and the period of delivery need not be very long, i.e., the period of constant delivery may be over a period of maybe half an hour or an hour or a few hours, but may also be over a period of days, weeks, months, or even years.

In preferred embodiments of the present invention, GDNF administration or delivery to target sites is achieved using pumps, catheters, and/or catheter systems to deliver exogenously produced GDNF to the brain. Certain embodiments of the present invention also contemplate methods wherein GDNF, GDNF producing and/or secreting agents are administered without the use of pumps, catheters, and/or catheter systems. It will be understood by those with skill in the art that, in the methods of the present invention administration of GDNF may be by way of alternative delivery methods well known in the art including, but not limited to, gene therapy using GDNF encoding genetic sequences or cellular therapy using cells capable of producing and secreting GDNF and subsequently resulting in the delivery of GDNF to target sites. Therapeutic delivery of GDNF to the target sites disclosed herein by gene or cellular means has the advantage of being less invasive than delivery of GDNF by infusion using pump and catheter systems.

Delivery of GDNF to the target sites disclosed herein may involve the introduction of nucleic acid (DNA or RNA) sequences encoding GDNF polypeptides into a patient to achieve expression of a GDNF polypeptide. Such gene therapy and gene delivery techniques are known in the art. As discussed more fully below, the GDNF encoding polynucleotide sequences preferably have a therapeutic effect after being taken up by a cell and wherein GDNF is subsequently expressed therein. A non-limiting example of polynucleotides that are therapeutic are DNA coding rRNA encoding GDNF. Thus, for example, cells from a patient may be engineered with a polynucleotide (DNA or RNA) comprising a GDNF encoding polynucleotide operably linked to a promoter ex vivo, with the engineered cells then being provided to a patient at the target sites disclosed herein subsequently resulting in the GDNF polypeptide being administered to such target sites. Such methods are well-known in the art. For example, see Santodonato, L., et al., Human Gene Therapy 7:1-10 (1996); Santodonato, L., et al., Gene Therapy 4:1246-1255 (1997); and Zhang, J.-F. et al., Cancer Gene Therapy 3: 31-38 (1996)). These engineered cells may be delivered to the target sites disclosed herein by various means known in the art including, but not limited to, direct injection or catheter injection.

Transplantation of cells and tissues is being utilized therapeutically in a wide range of disorders including neurodegenerative disorders. In some embodiments of the present invention, the GDNF is delivered to the target sites disclosed herein by use of transplanted cells or tissues. In one embodiment the methods of Freed, C. et al., (Freed, C. et al., 2001) are modified such that cultured embryonic mesencephalic tissue is transplanted to the targets sites disclosed herein. In another embodiment, the patient's own cells are engineered or induced to produce GDNF by transfection in vivo with a DNA that encodes GDNF. GDNF encoding DNA can be introduced into the patient's cells in vivo or ex vivo, for example, by injecting naked DNA or liposome-encapsulated DNA that encodes GDNF, or by other means of transfection. In another embodiment, the patient's own cells are engineered or induced to produce GDNF by transfection ex vivo with a DNA that encodes GDNF. Such cells are then delivered by any appropriate means to the target sites disclosed herein. In another embodiment of the present invention GDNF is delivered to the target sites disclosed herein in accordance with any one of the methods disclosed in U.S. Pat. No. 6,649,160 (Sanberg, et al.), the entire disclosure of which is hereby incorporated by reference into this specification. It is contemplated herein that GDNF can be delivered to the target sites disclosed herein by introducing GDNF encoding DNA into the patient's cells, for example, by injecting “naked” polynucleotides or liposome-encapsulated polynucleotides that encode GDNF, or by other means of transfection known in the art. By “naked” polynucleotides is meant that the polynucleotides are free from any delivery vehicle that acts to assist, promote, or facilitate entry into the cell, including viral sequences, viral particles, liposome formulation, lipofectin, precipitating agents and the like. Such methods are well known in the art and described, for example, in U.S. Pat. Nos. 5,593,972, 5,589,466, and 5,580,859.

The naked polynucleotides used in embodiments of the invention can be those which do not integrate into the genome of the host cell. These may be non-replicating sequences, or specific replicating sequences genetically engineered to lack the genome-integration ability. Alternatively, the naked polynucleotides used in the invention may integrate into the genome of the host cell by, for example, homologous recombination, as discussed below. Preferably, the naked GDNF polynucleotide construct is contained in a plasmid. Suitable expression vectors for include, but are not limited to, vectors such as pRSVcat (ATCC 37152), pSVL and MSG (Pharmacia, Uppsala, Sweden), pSV2dhfr (ATCC 37146) and pBC12MI (ATCC 67109). The naked polynucleotides can be administered to the target sites disclosed herein by any method known in the art, including, but not limited to, direct needle injection at the target site, catheter infusion, or so-called “gene guns”. These delivery methods are known in the art and discussed in more detail below.

For naked polynucleotide injection, an effective dosage amount of polynucleotide will be in the range of from about 0.05 μg/kg body weight to about 50 mg/kg body weight. Preferably, the dosage will be from about 0.005 mg/kg to about 20 mg/kg and more preferably from about 0.05 mg/kg to about 5 mg/kg. The appropriate and effective dosage of the polynucleotide construct can readily be determined by those of ordinary skill in the art.

The constructs encoding GDNF may also be delivered with delivery vehicles such as viral sequences, viral particles, liposome formulations, lipofectin, precipitating agents, etc. Such methods of delivery are known in the art. In certain embodiments, the GDNF encoding polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416) and mRNA (Malone et al., Proc. Natl. Acad. Sci. USA (I 989) 86:6077-6081) in functional form. Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Feigner et al., Proc. Natl. Acad. Sci. USA (1987) 84:7413-7416). Other commercially available liposomes include transfectace (DDAB/DOPE) and DOTAP/DOPE (Boehringer). Other cationic liposomes can be prepared from readily available materials using techniques well known in the art. See, e.g. PCT Publication No. WO 90/11092 for a description of the synthesis of DOTAP (1,2-bis(oleoyloxy)-3-(trimethylammonio)propane) liposomes. Preparation of DOTMA liposomes is explained in the literature, see, e.g., P. Feigner et al., Proc. Natl. Acad. Sci. USA 84:7413-7417. Similar methods can be used to prepare liposomes from other cationic lipid materials. Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art. For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15° C. Alternatively, negatively charged vesicles can be prepared without sonication to produce multilamellar vesicles or by extrusion through nucleopore membranes to produce unilamellar vesicles of discrete size. Other methods are known and available to those of skill in the art. The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983), 101:512-527. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²+-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166).

Additional examples of useful cationic lipids include dipalmitoyl-phophatidylethanolamine 5-carboxyspen-nylamide (DPPES); 5-carboxyspermylglycine dioctadecylamide (DOGS); dimethyldioctdecyl-ammonium bromide (DDAB); and (±)-N,N-dimethyl-N-[2-(sperminecarboxamido)ethyl]-2,3-bis(dioleyloxy)-1-propaniminium pentahydrochloride (DOSPA). Non-diether cationic lipids, such as DL-1,2-dioleoyl-3-dimethylaminopropyl-β-hydroxyethylammonium (DORI diester), 1,2-O-dioleyl-3-dimethylaminopropyl-β-hydroxyethylammonium (DORIE diether), 1-O-oleyl-2-oleoyl-3-dimethylaminopropyl-β-hydroxyethylammonium (DORI ester/ether), and their salts promote in vivo gene delivery. Cationic cholesterol derivatives such as, {3β[N-N′,N′-dimethylamino)ethane]-carbomoyl}-cholesterol (DC-Chol), are also useful. Preferred cationic lipids include: (±)-N-(2-hydroxyethyl)-N,N-dimethyl-2,3-bis(tetradecyloxy)-1-propaniminium bromide; 3,5-(N,N-di-lysyl)diaminobenzoylglycyl-3-(DL-1,2-dioleoyl-dimethylaminopropyl-β-hydroxyethylamine) (DLYS-DABA-GLY-DORI diester); 3,5-(NN-dilysyl)-diaminobenzoyl-3-(DL-1,2-dioleoyl-dimethylaminopropyl-β-hydroxyethylamine) (DLYS-DABA-DORI diester); and 1,2-dioleoyl-sn-glycero-3-phosphoethanolamine. Also preferred is the combinations of the following lipids: (±)-N-(2-hydroxyethyl)-N,N-dimethyl-2,3-bis(tetradecyloxy)-1-propaniminium bromide and 1,2-dioleoyl-sn-glycero-3-phosphoethanolamine; and (±)-N-(2-hydroxyethyl)-N,N-dimethyl-2,3-bis(tetradecyloxy)-1-propaniminium bromide, and 1,2-dioleoyl-sn-glycero-3-phosphoethanolamine in a 1:1 ratio. The lipid formulations may have a cationic lipid alone, or also include a neutral lipid such as cardiolipin, phosphatidylcholine, phosphatidylethanolamine, dioleoylphosphatylcholine, dioleoylphosphatidyl-ethanolamine, 1,2-dioleoyl-sn-glycero-3-phosphatidylethanolamine (DOPE), sphingomyelin, and mono-, di- or tri-acylglycerol). Lipid formulations may also have cationic lipid together with a lysophosphatide. The lysophosphatide may have a neutral or a negative head group. Useful lysophosphatides include lysophosphatidylcholine, lysophosphatidyl-ethanolamine, and 1-oleoyl lysophosphatidylcholine. Lysophosphatide lipids are present. Other additives, such as cholesterol, fatty acid, ganglioside, glycolipid, neobee, niosome, prostaglandin, sphingolipid, and any other natural or synthetic amphiphiles, can be used. A preferred molar ratio of cationic lipid to neutral lipid in these lipid formulations is from about 9:1 to about 1:9; an equimolar ratio is more preferred in the lipid-containing formulation in a 1:2 ratio of lysolipid to cationic lipid.

Generally, the ratio of DNA to liposomes will be from about 10:1 to about 1:10. Preferably, the ratio will be from about 5:1 to about 1:5. More preferably, the ratio will be about 3:1 to about 1:3. Still more preferably, the ratio will be about 1:1.

U.S. Pat. No. 5,676,954 describes the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and International Publication No. WO 94/29469 provide cationic lipids for use in transfecting DNA into cells and mammals. U.S. Pat. Nos. 5,589,466, 5,693,622, 5,580,859, 5,703,055, and International Publication No. WO 94/29469 provide methods for delivering DNA-cationic lipid complexes to mammals.

Nucleic acids of the invention can also be administered to the target sites disclosed herein by other known methods for introduction of nucleic acid into a cell or organism including, without limitation, by infecting the brain with live replication deficient viral particles engineered to deliver GDNF (see, for example, Bjorklund, et al., 2000(a); Bjorklund, et al., 2000(b); Gash, et al., 1998; Grondin, et al., 2002b; Kordower, et al., 2000; Kordower, J. H., 2003; and U.S. Pat. Nos. 6,713,293, 6,683,058, U.S. Patent Application No: US20020187951 (Aebischer et al.), and International Patent Application Publication No: WO 97/39629. The entire disclosure of each of these references is hereby incorporated by reference into this specification. In certain other embodiments, cells are engineered, ex vivo or in vivo, with the GDNF polynucleotide operably linked to a promoter contained in an adenovirus vector. Adenovirus can be manipulated such that it encodes and expresses the desired gene product, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Suitable adenoviral vectors useful in the present invention are known in the art. Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, for example, the GDNF polynucleotide which would be used in these embodiments of the present invention, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles. It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377. For example, an appropriate AAV vector for use in the present invention will include all the sequences necessary for DNA replication, encapsidation, and host-cell integration. The GDNF polynucleotide construct is inserted into the AAV vector using standard cloning methods. The recombinant AAV vector is then transfected into packaging cells which are infected with a helper virus, using any standard technique, including lipofection, electroporation, calcium phosphate precipitation, etc. Appropriate helper viruses include adenoviruses, cytomegalo-viruses, vaccinia viruses, or herpes viruses. Once the packaging cells are transfected and infected, they will produce infectious AAV viral particles which contain the GDNF polynucleotide construct. These viral particles are then used to transduce eukaryotic cells, either ex vivo or in vivo at one or more target sites disclosed herein. The transduced cells will contain the GDNF polynucleotide construct integrated into its genome, and will express the molecule of interest.

At some target sites it may be preferable to use methods other than viral vectors to deliver the GDNF. Viral vectors may spread to other areas of the brain that are not to be targeted and it may be preferable to avoid this.

Another method of gene therapy which may be used to administer GDNF in the methods of the present invention involves operably associating heterologous control regions (e.g., a promoter of interest) and endogenous polynucleotide sequences (e.g., GDNF) via homologous recombination (see, e.g., U.S. Pat. No. 5,641,670; International Publication No. WO 96/29411; and International Publication No. WO 94/12650). This method involves the activation of a gene which is present in the target cells, but which is not normally expressed in the cells, or is expressed at a lower level than desired. Polynucleotide constructs are made, using standard techniques known in the art, which contain the promoter of interest with targeting sequences flanking the promoter of interest. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together. The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The promoter-targeting sequence can be delivered by any method, included direct needle injection, catheter injection, catheter infusion, particle accelerators, etc. The methods are described in more detail below. The promoter-targeting sequence construct is taken up by cells. Homologous recombination between the construct and the endogenous sequence takes place, such that an endogenous sequence (e.g., GDNF) is placed under the control of the promoter.

The promoter then drives the expression of the endogenous sequence (e.g., GDNF).

Any mode of administration to the target sites disclosed herein of any of the above-described polynucleotides constructs can be used so long as the mode results in the expression of GDNF in an amount sufficient to provide a therapeutic effect. This includes direct needle injection, catheter injection infusion, particle accelerators (i.e., “gene guns”), gelfoam sponge depots, other depot materials, osmotic pumps (e.g., Alza minipumps), or suppositorial solids. A preferred method of administration is by direct injection into the target site or catheter injection, i.e., injection into a catheter with its distal end positioned so as to allow for the delivery of the substance to at least one of the target sites disclosed herein.

A pharmaceutically or therapeutically effective amount of a substance is the amount required to bring about the desired pharmaceutical or therapeutic effect.

Determining an effective amount of substance to be delivered can depend upon a number of factors including, for example, the chemical structure and biological activity of the substance, the age and weight of the patient, and the severity of the disorder being treated. The frequency of treatments depends upon a number of factors, as well, such as the amount of polynucleotide constructs administered per dose, as well as the health and history of the subject. The precise amount, number of doses, and timing of doses will be determined by the attending physician.

In some embodiments of the present invention, the GDNF delivery device may be a device such as is described in U.S. Pat. No. 4,892,538 (Aebischer et al.) and U.S. Patent Application No. US20020150603 (Aebischer et al.) which are both incorporated herein by reference in their entireties. An example of a preferred delivery device for administration of GDNF to the target site is a device having a semipermeable wall enclosing encapsulated cells capable of secreting the GDNF. Alternatively, an device could consist of a polymeric insert with the GDNF or GDNF producing cells or vectors embedded within or on the surface of the polymer in such a way as to be slowly administer GDNF to the target site over time. Such a device is described in U.S. Pat. Nos. 4,346,709 (Schmitt) and 5,330,768 (Yamahira et al.), both of which are hereby incorporated by reference.

“Admixing” as used herein denotes the addition of an excipient to a polypeptide of interest, such as by mixing of dry reagents or mixing of a dry reagent with a reagent in solution or suspension, or mixing of aqueous formulations of reagents.

“Excipient” as used herein denotes a non-therapeutic agent added to a pharmaceutical composition to provide a desired consistency or stabilizing effect.

“Implanted” means placed within the body, and maintained at that location for some extended period of time. As used herein it is intended that the period of time during which the implanted object is maintained in place will be, in general, considerably greater than that customarily required to introduce a bolus of a substance, such as a drug. For example, a catheter used in a method of the invention may be placed within a tissue or organ such that the catheter so implanted is intended to remain at the site of implantation for some extended period of time. Some of the drug delivery apparatuses that may be used in the methods of the invention, for example the drug pumps and/or catheters, are designed to be implanted for periods greater than a month and even years and to deliver drug during this period. A drug delivery apparatus may be implanted, for example, subcutaneously, or within a tissue or organ, or within a body cavity such as the peritoneal cavity, infraclavicular space, the thoracic cavity, the pelvic cavity, or any other cavity or location that is convenient for delivery of the intended substance. A catheter may be implanted into a tissue, for example, brain tissue, and may be affixed in place by fixing the catheter to another tissue, such as bone, e.g., the skull, or cartilage, using an adhesive or screws, clamps, sutures, or any other suitable fixing means.

The phrases “dopaminergic dysfunction”, “dopaminergic dysregulation”, “dopaminergic degeneration”, “dopamine depleted”, “dopamine deficient”, or grammatical equivalents thereof, may be used interchangeably herein. All such phrases are intended to encompass at least one of the following conditions or disorders: Parkinson's disease, neuronal dopamine deficit, dopaminergic neuron deficit, dopaminergic neuron lesions, hypo-dopaminergic innervation, dopamine synthesis incapacity, dopamine storage incapacity, dopamine transport incapacity, or dopamine uptake incapacity. Dopaminergic dysfunction can be evidenced by analyzing factors including, but not limited to, the following: 1) the number of TH expressing neurons 2) size of dopamine neuronal cells 3) dopamine metabolite levels 4) dopamine uptake, 5) dopamine transport, 6) neuronal dopamine uptake, 7) dopamine transporter binding, 8) quantal size of terminal dopamine release, 9) rate of dopamine turnover, 10) TH+ cell count, 11) TH+ innervation density and 12) TH+ fiber density.

The phrase “target site”, or a grammatical version thereof, refers to the site for intended delivery of a substance, such as GDNF. In particular embodiments of the present invention, the preferred target site is at least one SNc and a subthalamic region of the brain of a human afflicted with PD. More preferably, the target site is the SNc and/or STN. Even more preferably, the target site is the SNc and/or ZI. Even more preferably, the target site is the SNc and/or caudal ZI. Even more preferably, at least one putaminal region are the target sites. Even more preferably, at least one putaminal region and the SNc and the ZI are target sites. Even more preferably, at least one putaminal region and the SNc and/or the caudal ZI are target sites. Even more preferably, the SNc, the caudal ZI and at least one putaminal region are target sites. Even more preferably, the target site is the SNc and the ZI. Even more preferably, at least one putaminal region and at least one of the lateral SNc and the STN are the target sites. Even more preferably, at least one putaminal region are target sites. Even more preferably, the lateral SNc, the ZI and at least one putaminal region and at least one of the lateral SNc and the ZI are target sites. Even more preferably, the lateral SNc, the caudal ZI and at least one putaminal region are target sites. Even more preferably, the lateral SNc, the caudal ZI and the dorsal putamen are target sites. Even more preferably, the lateral SNc, the caudal ZI and the central region of the putamen are target sites. Most preferably, the lateral SNc, the caudal ZI and the posterodorsal putamen are target sites. Furthermore, any particular target site may be targeted unilaterally or bilaterally with respect to the hemispheres of the brain.

“Proximal end” is a relative term, and generally refers to the end of a device, such as a catheter that is nearest to the operator (i.e., the surgeon) and is furthest away from the treatment site. In the present invention a catheter has a proximal end that may be communicably attached to an access port or drug delivery apparatus, such as a pump, or reservoir.

“Tyrosine hydroxylase-positive” or “TH+” is intended to refer to the presence of tyrosine hydroxylase in a referenced nervous tissue as indicated by the results from any technique known in the art as a means to detect and/or measure tyrosine hydroxylase, tyrosine hydroxylase encoding mRNA, or tyrosine hydroxylase activity.

“Distal end” is a relative term and generally refers to the end of a device, such as a catheter, that is furthest away from the operator (i.e., the surgeon) and is closest to the treatment site. In the present invention the distal end of a catheter may be communicably attached to an opening that allows for the delivery of drug to the target site.

“Drug delivery apparatus” as used herein includes but is not limited to, a drug reservoir and/or a drug pump of any kind, for example an osmotic pump, an electromechanical pump, an electro-osmotic pump, an effervescent pump, a hydraulic pump, a piezoelectric pump, an elastomeric pump, a vapor pressure pump, or an electrolytic pump. Preferably, such a pump is implanted within the body.

Throughout this specification, reference to the term “GDNF” or the phrase “GDNF protein product” or “GDNF polypeptide”, all of which are used interchangeably, refers to glial cell line-derived neurotrophic factor from any species, including murine, bovine, ovine, porcine, equine, avian, and preferably human, in native sequence or in genetically engineered variant form, including, without limitation, biologically active fragments, analogs, variants, (including, insertion, substitution, and deletion variants) and derivatives thereof, and from any source, whether natural, synthetic, or recombinantly produced. A “biologically effective fragment” is a fragment which is able to function in the same way as GDNF.

Exemplary GDNF polypeptides useful in the methods of the present invention include, without limitation, any of GDNF protein products described in U.S. Pat. Nos. 5,731,284, 6,362,319, 6,093,802, and 6,184,200 (all of which are hereby incorporated by reference in their entireties). Preferred GDNF protein products for use in the methods of the present invention include, but are not limited to, r-metHuGDNF, a recombinant GDNF protein produced in E. coli which has an amino acid sequence identical to native mature human GDNF with the addition of an amino terminal methionine. Thus, r-metHuGDNF consists of 135 amino acids. Seven of the amino acids are cysteines, which are involved in one intermolecular disulfide bond and three intramolecular disulfide bonds. In its active form, r-metHuGDNF is a disulfide-bonded homodimer. The primary amino acid sequence of monomeric r-metHuGDNF is provided in Table 1.

TABLE 1
Primary Amino Acid Sequence of r-metHuGDNF

| | |
|---|---|
| | Amino |
| | Acid |
| Primary Amino Acid Sequence (SEQ ID NO:1) | No. |

| | | |
|---|---|---|
| H₂N-Met | Ser Pro Asp Lys Gln Met Ala Val Leu Pro | 10 |
| | Arg Arg Glu Arg Asn Arg Gln Ala Ala Ala | 20 |
| | Ala Asn Pro Glu Asn Ser Arg Gly Lys Gly | 30 |
| | Arg Arg Gly Gln Arg Gly Lys Asn Arg Gly | 40 |
| | Cys Val Leu Thr Ala Ile His Leu Asn Val | 50 |
| | Thr Asp Leu Gly Leu Gly Tyr Glu Thr Lys | 60 |
| | Glu Glu Leu Ile Phe Arg Tyr Cys Ser Gly | 70 |
| | Ser Cys Asp Ala Ala Glu Thr Thr Tyr Asp | 80 |
| | Lys Ile Leu Lys Asn Leu Ser Arg Asn Arg | 90 |
| | Arg Leu Val Ser Asp Lys Val Gly Gln Ala | 100 |
| | Cys Cys Arg Pro Ile Ala Phe Asp Asp Asp | 110 |
| | Leu Ser Phe Leu Asp Asp Asn Leu Val Tyr | 120 |
| | His Ile Leu Arg Lys His Ser Ala Lys Arg | 130 |
| | Cys Gly Cys Ile COOH | 134 |

The GDNF protein products useful in the present invention may be isolated or generated by any means known to those skilled in the art. Preferably, GDNF is recombinantly produced. In a preferred method, the GDNF is cloned and its DNA expressed, e.g., in mammalian cells or bacterial cells. Exemplary methods for producing GDNF protein products useful in the present invention are described in U.S. Pat. Nos. 6,362,319, 6,093,802 and 6,184,200 (all of which are hereby incorporated by reference in their entireties).

GDNF pharmaceutical compositions typically comprise a therapeutically effective amount of at least one GDNF protein product and one or more pharmaceutically and physiologically acceptable formulation agents. Suitable formulation agents include, but are not limited to, antioxidants, preservatives, coloring, flavoring and diluting agents, emulsifying agents, suspending agents, solvents, fillers, bulking agents, buffers, vehicles, diluents, excipients and/or pharmaceutical adjuvants. For example, a suitable vehicle may be, physiological saline solution, citrate buffered saline, or artificial CSF, possibly supplemented with other materials common in compositions for parenteral administration. Neutral buffered saline or saline mixed with serum albumin are further exemplary vehicles. Those skilled in the art would readily recognize a variety of buffers that could be used in the compositions, and dosage forms used in the invention. Typical buffers include, but are not limited to pharmaceutically acceptable weak acids, weak bases, or mixtures thereof. Preferably, the buffer components are water soluble materials such as phosphoric acid, tartaric acids, lactic acid, succinic acid, citric acid, acetic acid, ascorbic acid, aspartic acid, glutamic acid, and salts thereof.

The primary solvent in a vehicle may be either aqueous or non-aqueous in nature. In addition, the vehicle may contain other pharmaceutically-acceptable excipients for modifying or maintaining the pH, osmolarity, viscosity, clarity, color, sterility, stability, rate of dissolution, or odor of the formulation. A preferred pharmaceutical composition of GDNF comprises a therapeutically effective amount of at least one GDNF protein and a pharmaceutically acceptable vehicle. More preferably, the pharmaceutically acceptable vehicle is an aqueous buffer. More preferably, the vehicle comprises sodium chloride at a concentration of about 100 mM to about 200 mM and sodium citrate at a concentration of about 5 mM to about 20 mM. Even more preferably, the vehicle comprises sodium chloride at a concentration of about 125 mM to about 175 mM and sodium citrate at a concentration of about 7.5 mM to about 15 mM. Even more preferably, the vehicle comprises sodium chloride and sodium citrate at a concentration of about 150 mM and about 10 mM, respectively. Even more preferably, the vehicle comprises sodium chloride and sodium citrate at a concentration of about 120 mM and about 10 mM, respectively. Even more preferably, the GDNF pharmaceutical composition is formulated as a liquid with a pH of around 5.0 to around 5.5. Most preferably, the GDNF pharmaceutical composition is formulated as a liquid with 10 mM sodium citrate and 150 mM sodium chloride with a pH of 5.0.

The GDNF pharmaceutical composition may contain still other pharmaceutically-acceptable formulation agents for modifying or maintaining the rate of release of GDNF protein product. Such formulation agents are those substances known to artisans skilled in formulating sustained release formulations. For further reference pertaining to pharmaceutically and physiologically acceptable formulation agents, see, for example, Remington's Pharmaceutical Sciences, 18th Ed. (1990, Mack Publishing Co., Easton, Pa. 18042) pages 1435-1712 (the disclosure of which is hereby incorporated by reference).

Once the therapeutic composition has been formulated, it may be stored in sterile vials as a solution, suspension, gel, emulsion, solid, or dehydrated or lyophilized powder. Such formulations may be stored either in a ready to use form, a lyophilized form requiring reconstitution prior to use, or a liquid form requiring dilution prior to use. Preferably, the GDNF pharmaceutical composition is provided in sterile single-use vials at a concentration of 10 mg/mL and stored at a temperature of 2-8° C. until use. Immediately prior to administration, the GDNF protein product is appropriately diluted with an appropriate sterile citrate buffered saline such as any of those discussed above.

In primates, direct infusion into the SNc has been shown to increase dopamine delivery to the striatum by up to 200% with improvement in experimental Parkinsonism. However, in Parkinson's disease, degeneration occurs in dopamine neurons involving relatively dispersed nuclei in the mid brain and the degree of degeneration in specific nuclei may reflect the clinical presentation (Grondin, et al., 2002a; Grondin, et al., 2003).

Degeneration involving the lateral part of the SNc (also called lateral area A9 of Dahlstrom and Fuxe) which supplies the motor striatum and GPe is associated with akinetic/bradykinetic PD, whereas degeneration involving the medial SNc (medial area A9), parts of area A8 and A10 which innervate the STN/Zona Incerta and GPi is associated with tremor predominant PD and “on-off” fluctuations. (Francois, C., et al., 1999; Francois, C., et al., 2000; Hassani, O. K., et al., 1997; Smith, Y., et al., 2000; Kolmac, C., et al., 1998; Ka, J., 2002; Jellinger, K., et al., 1980) (see FIG. 1). In preferred embodiments of the present invention, a single intraparenchymal catheter is strategically placed with the tip immediately above the lateral A9 area in the caudal ZI. GDNF then may diffuse away from the catheter tip (up to several millimeters from the catheter tip) into the STN/ZI to be taken up by existing dopamine terminals and retrogradely transported to medial SNc (medial area A9), area A8, and A10, enhancing their dopamine delivery to the STN/ZI as well as GPi. Consequently, terminal sprouting is most likely to occur in STN/ZI but not GPi. Axons from these neurons have wide terminal fields in the striatum which may lead to a more even and more widespread delivery of dopamine than has been achieved by prior methods which rely on the direct infusion of GDNF solely to the posterior dorsal putamen (e.g., the putamen) as reported by Gill, et al. (Gill, et al., 2003) or the SNr (Elsberry et al., U.S. Pat. No. 6,042,579). Furthermore, dopaminergic innervation of the subthalamic nucleus is presynaptic and has a role in modulating glutamate release to STN neurons (Magill, P. J., et al., 2001). In Parkinson's disease loss of dopamine will result in excessive release of glutamate from its afferents (cortical and CMIPF drivers). STN neurons will tend to burst fire at fronto-cortical frequency of about 20 Hz. As each STN neuron innervates multiple neurons in the basal ganglia output nuclei (Gpi and SNr), the bursts will generate synchronised oscillations in them. In tremor predominant PD, low frequency (20 Hz) synchronised oscillations have been identified in Gpi and STN neurons, as well as the typical 5 Hz oscillation (Brown, P., et al, 2001; Levy, R., et al., 2002). Loss of dopamine to the ZI will also cause them to burst fire and oscillate (Perier, C., et al., 2000). The ZI receives input from the basal ganglia, the ascending reticular activating system (RAS), the cerebellar interpositus nucleus and associative and limbic areas which facilitate and modulate motor behaviour (Kolmac, C. I., Power, B. D., Mitrofanis, J., 1998; Roger, M. and Cadusseau, J., 1985; Mitrofanis, J. and deFonseka, R., 2001). It has excitatory output to the CM/Pf which synchronises neuronal firing in the striatum and the STN (Power, B. D., et al., 2002; Lin, C. S., et al., 1990). It also feeds out directly to the cortex and locomotor centres in the brainstem (Lin, C. S., et al., 1990). The altered firing patterns in ZI neurons resulting from dopamine depletion is likely to have a profound effect on motor function. And finally, in idiopathic PD, in addition to degeneration of dopamine neurons in SNc and area A8, cholinergic neurons in PPN, noradrenergic neurons in locus coerulus and midbrain serotonergic neurons have all been seen to degenerate in parallel to a variable degree (Paulus, W. J. K., 1991). In a PET study by Doder, et al., tremor severity in PD was correlated with midbrain serotonin depletion (Doder et al., 2003). Serotonin is known to raise the firing threshold of STN/ZI neurons. Therefore, if serotonin neurons degenerate in parallel with dopaminergic neurons there may be a synergistic effect in provoking STN/ZI neurons to burst fire. GDNF has recently been shown to protect and up regulate serotonergic and noradrenergic neurons. Therefore, direct infusion of GDNF into STN/ZI may also restore the integrity of these neuronal modulators. It follows that reducing dopamine deficiencies in subthalamic regions and, more particularly, the caudal ZI/STN may lead to significant improvements in symptomatic control of PD.

Accordingly, the present invention is based on the concept that continuous delivery of GDNF directly to at least one of the SNc and a subthalamic region of the brain or, more preferably, to at least one putaminal region and at least one of the SNc and at least one subthalamic region of the brain, by means of an implantable pump and at least one indwelling catheter in human PD patients will result in anti-parkinsonian and anti-dyskinetic effects, including, but not limited to, improvement in tremor and/or dystonia, in addition to the re-innervation and restoration of critically important neurotransmitter stores in previously neurotransmitter deficient neurons.

In a particularly preferred embodiment of the present invention the GDNF is delivered to the putaminal region by way of a pump or a catheter implanted in that region. It is advantageous to use a pump or catheter to deliver the drug to that area because other methods of delivery may not allow sufficient volumes of the drug to be delivered. In contrast, GDNF may be easily delivered to other target sites, in particular the SNc or subthalamic regions by using, for example, encapsulated cells, stem cells or viral vectors, as these sites may require a lower volume of drug to be delivered to a more localized area.

In a particularly preferred embodiment of the present invention GDNF is delivered both to the putamen and to the subthalamic region such as the substantia nigra, zona incerta and the subthalamic nucleus. Delivery of GDNF to the putamen alone may improve some aspects of Parkinson's disease including bradykinesia, but may not improve tremor which results from loss of dopamine and probably serotonin innervation of the subthalamic nucleus and the zona incerta. Dual delivery is therefore likely to be more beneficial in achieving symptomatic control. When axonal sprouting and reinnervation of the putamen has been achieved then it may be possible to discontinue treatment of the putamen and maintain putamenal dopaminergic innovation by delivering GDNF to the substantia nigra compacta alone. In order to treat the relatively large volume of the posterior third of the putamen, a preferred method is to implant a catheter and deliver GDNF by a pump. With an appropriately designed catheter and infusion regime the posterior third of the putamen can be treated via a single catheter using the technique of “convection enhanced delivery”. Drug emanating from a catheter port implanted in tissues will tend to follow the path of least resistance and this is typically along the catheter tissue interface. Resistance in this pathway is inversely proportional to the radius of a catheter squared and so by reducing the catheter dimensions to less than 1 mm and preferably less than 0.6 mm, resistance in this pathway can be increased to the point where the drug will preferentially be driven into the tissues. The volume of delivery is then proportional to the pressure gradient developed up to the point where the pressure overcomes the resistive pathway along the catheter tissue interface. Using a catheter with an outside diameter of 0.6 mm and a flow rate of between 6 and 12 μg per hour the infusate will be distributed throughout the posterior third of the dorsal putamen if the catheter is appropriately sited in the middle of this volume. Achieving safe and/or adequate distribution of GDNF to the posterior third of the putamen using viral vectors or stem cells manufacturing GDNF is more difficult. Although viral vectors could be delivered by convection enhanced delivery it is not possible to contain them within the planned treatment volume because the infusate will, for example, be carried along perivascular spaces to the cerebral spinal fluid and from there viral vectors may be carried to distant sites in the central nervous system. In order to treat the relatively large volume of tissue in the posterior putamen with GDNF using stem cells a homogenous distribution of cells throughout the volume is necessary. If the cells are encapsulated then multiple implants will be required to achieve sufficient distribution of GDNF throughout the tissue. This is because the quantity of drug manufactured by the cells is in order of nanograms and its distribution is dependent upon establishing a concentration gradient which, with such low concentrations will not allow adequate diffusion of GDNF. The most practicable means to deliver GDNF to the posterior putamen is therefore with a pump and catheter with an infusion regime and a catheter designed for convection enhanced delivery. By contrast the volume of distribution required to treat the subthalamic region including the substantia nigra compacta, the zona incerta and the subthalamic region is only a few millimetres and within the range of diffusion of encapsulated cells. Treatment with encapsulated cells has the advantage that the GDNF is manufactured in situ and the patient does not require repeated refills of a pump which may be on a monthly basis. A preferred treatment regime would therefore be to surgically implant a catheter into each posterior putamen which are in turn connected to pumps before continuous or pulse delivery of GDNF to achieve convection enhanced delivery to the desired volumes. At the same surgery encapsulated cells manufacturing GDNF could also be implanted into the subthalamic region such that their volume of delivery will encompass the lateral portion of the substantia nigra compacta, the zona incerta and the subthalamic nucleus. When reinnervation of the putamen has been achieved using convection enhanced delivery of GDNF, a process which may take two to three years, then infusion may be discontinued and the integrity of the nigral striatal neurons and their regenerated axons would be maintained by continued delivery of GDNF from the implanted encapsulated cells.

The methods of the present invention are contemplated as being useful in repairing neural pathways damaged by Parkinson's disease in humans. Specifically, the methods described herein are capable of stimulating nerve regeneration, including re-innervation of damaged human brain tissue by neurons including, but not limited to, dopaminergic neurons. In a preferred embodiment there is provided a method of stimulating nerve regeneration, including re-innervation of damaged human brain tissue by neurons including, but not limited to, serotonergic neurons that comprises administering a pharmaceutically effective dose of r-metHuGDNF to at least one of the SNc and a subthalamic region or, alternatively at least one putaminal region and at least one of the SNc and a subthalamic region of the brain of a human patient in need thereof. In a more preferred embodiment there is provided a method of stimulating nerve regeneration, including re-innervation of damaged human brain tissue by neurons including, but not limited to, dopaminergic and noradrenergic neurons that comprises administering a pharmaceutically effective dose of r-metHuGDNF to at least one of the SNc and a subthalamic region or, alternatively, at least one putaminal region and at least one of the SNc and a subthalamic region of the brain of a human patient in need thereof. In an even more preferred embodiment there is provided a method of stimulating nerve regeneration, including re-innervation of damaged human brain tissue by neurons including, but not limited to, dopaminergic and serotonergic neurons that comprises administering a pharmaceutically effective dose of r-metHuGDNF to at least one of the SNc and a subthalamic region or, alternatively, at least one putaminal region and at least one of the SNc and a subthalamic region of the brain of a human patient in need thereof. In an even more preferred embodiment there is provided a method of stimulating nerve regeneration, including re-innervation of damaged human brain tissue by neurons including, but not limited to, dopaminergic, serotonergic, cholinergic, and noradrenergic neurons that comprises administering a pharmaceutically effective dose of r-metHuGDNF to at least one of the SNc and a subthalamic region or, alternatively, at least one putaminal region and at least one of the SNc and a subthalamic region of the brain of a human patient in need thereof. In a most preferred embodiment of the present invention there is provided a method of stimulating nerve regeneration, including re-innervation of damaged human brain tissue by neurons including, but not limited to, dopaminergic, serotonergic, cholinergic, and noradrenergic neurons that comprises administering a pharmaceutically effective dose of r-metHuGDNF to at least one of the SNc, preferably, the lateral SNc, and at least one subthalamic region, preferably the caudal zona incerta or, alternatively, at least one putaminal region, preferably the posterodorsal putamen, and at least one of the SNc, preferably, the lateral SNc, at least one subthalamic region, preferably, the caudal zona incerta of the brain of a human patient in need thereof.

The present invention also concerns the use of a pharmaceutically effective amount of GDNF and at least one pharmaceutically acceptable vehicle, excipient, or diluent in the preparation of a pharmaceutical composition for increasing the function of neurons rendered dysfunctional by Parkinson's disease, wherein the composition is for administration to at least one of the SNc and a subthalamic region or, alternatively, to at least one putaminal region and at least one of the SNc and a subthalamic region of the brain of a human patient in need thereof. Therefore, the methods of the present invention are contemplated as being useful in increasing the function of neurons which are affected by Parkinson's disease in humans. In a preferred embodiment there is provided a method of increasing the function of dopaminergic neurons that comprises administering a pharmaceutically effective dose of r-metHuGDNF to at least one of the SNc and a subthalamic region or, alternatively, to at least one putaminal region and at least one of the SNc and a subthalamic region of the brain of a human patient in need thereof. In a more preferred embodiment there is provided a method of increasing the function of dopaminergic and noradrenergic neurons that comprises administering a pharmaceutically effective dose of r-metHuGDNF to at least one of the SNc and a subthalamic region or, alternatively, to at least one putaminal region and at least one of the SNc and a subthalamic region of the brain of a human patient in need thereof. In an even more preferred embodiment there is provided a method of increasing the function of dopaminergic and serotonergic neurons that comprises administering a pharmaceutically effective dose of r-metHuGDNF to at least one of the SNc and a subthalamic region or, alternatively, to at least one putaminal region and at least one of the SNc and a subthalamic region of the brain of a human patient in need thereof. In an even more preferred embodiment there is provided a method of increasing the function of dopaminergic, serotonergic, cholinergic, and noradrenergic neurons that comprises administering a pharmaceutically effective dose of r-metHuGDNF to at least one of the SNc and a subthalamic region or, alternatively at least one putaminal region and at least one of the SNc and a subthalamic region of the brain of a human patient in need thereof. In a most preferred embodiment of the present invention there is provided a method of increasing the function of dysfunctional neurons including, but not limited to, dopaminergic, serotonergic, cholinergic, and noradrenergic neurons that comprises administering a pharmaceutically effective dose of r-metHuGDNF at least one of the SNc, preferably, the lateral SNc, and a subthalamic region, preferably the caudal zona incerta or, alternatively, at least one subthalamic region, preferably, the caudal zona incerta, and at least one putaminal region, preferably the posterodorsal putamen and at least one of, of the brain of a human patient in need thereof.

In the methods of the present invention, GDNF is chronically administered to one or more target sites in the human brain by means of an implantable pump and one or more catheters. Preferably, one or more of the target sites is identified by assessing biomarkers of PD disease or disease progression including, but not limited to, the number of TH expressing neurons 2) size of dopamine neuronal cells 3) dopamine metabolite levels 4) dopamine storage, 5) dopamine transport, 6) neuronal dopamine uptake, 7) dopamine transporter binding, 8) quantal size of terminal dopamine release, 9) rate of dopamine turnover, 10) TH+ cell count, 11) TH+ innervation density and 12) TH+ fiber density. Even more preferably, one or more of the target sites is determined by neuroimagery of the brain, or regions thereof. Even more preferably, the neuroimagery technique used to determine one or more target sites for chronic infusion of GDNF is selected from the group consisting of ¹⁸F-fluorodopa positron emission tomography (¹⁸F-dopa PET)¹³ and ¹²³I-2β-carboxymethoxy-3β-(4-iodophenyl)tropane uptake on single-photon emission tomography (¹²³I-β-CIT SPECT). In an even more preferred embodiment of the present invention, GDNF is chronically infused directly into at least one dopaminergic dysfunctional putamen in addition to the lateral SNc and caudal zona incerta of a PD patient. Even more preferably, GDNF is chronically infused directly into central region of at least one dopaminergic dysfunctional putamen in addition to the lateral SNc and caudal zona incerta of a PD patient. Even more preferably, GDNF is chronically infused directly into the posterior region of at least one dopaminergic dysfunctional putamen in addition to the lateral SNc and caudal zona incerta of a PD patient. Most preferably, GDNF is chronically infused directly into at least one dopaminergic dysfunctional postero-dorsal putamen in addition to the lateral SNc and caudal zona incerta of a PD patient.

A number of drug delivery apparatus, catheters, catheter systems and combinations thereof have been developed for the dispensing of medical substances to specific sites within the body and are all readily available to those skilled in the art for use in the methods of the present invention. Therefore, one may use prior art drug delivery devices, catheters, and catheter systems for delivering the GDNF compositions to the target site of the brain of the patient at specified concentrations and/or at specified times and/or at different delivery rates. By way of illustration and not limitation, in the methods of the present invention one may use the technology described in U.S. Patent Publication No. US20030120262, US20030208184, or US20030225372 or U.S. Pat. No. 4,931,050, 4,838,887, 5,207,666, 4,714,462, 5176,641; 3,923,060, 4,003,379, 4,588,394, 4,447,224, 5,575,770, 4,978,338, 5,908,414, 5,643,207, 6,589,205 or 6,592,571. The entire disclosure of each of these U.S. patent applications and U.S. patents is hereby incorporated by reference into this specification. A preferred drug delivery apparatus useful in the context of the present invention includes one described in U.S. Pat. No. 5,752,930 or U.S. Patent Application No. US20030216714 (which are hereby incorporated by reference in their entireties). A more preferred drug delivery apparatus useful in the context of the present invention includes one described in U.S. Pat. No. 6,620,151 (which is hereby incorporated by reference in its entirety). An even more preferred drug delivery apparatus useful in the context of the present invention includes one described in U.S. Patent Application No. US20030216714 (which is hereby incorporated by reference in its entirety). Most preferably the drug delivery apparatus used in the context of the present invention is one described in U.S. Pat. Nos. 4,146,029, 4,013,074, or 4,692,147, (which are hereby incorporated by reference in their entireties) commercial embodiments thereof including, but not limited to, the Synchromed® I, Synchromed® EL, and Synchromed® II infusion pumps (Medtronic, Inc., Minneapolis, Minn.).

In another embodiment of the present invention, in conjunction with any of the above or below embodiments, a number of catheters and catheter systems have been developed for the dispensing of agents, such as drugs, to specific sites in the body and are readily available to those skilled in the art for use in the methods of the present invention. By way of illustration and not limitation, in the methods of the present invention one may use the technology described in U.S. Patent Publication No. 20030216700, 20030199831, or 20030199829 or U.S. Pat. No. 6,319,241. The entire disclosure of these U.S. patent applications and the United States patent is hereby incorporated by reference into this specification. A preferred catheter or catheter system useful in the context of the present invention includes, but is not limited to, an intraparenchymal infusion catheter or catheter system described in International Patent Application Publication No: WO 02/07810 or WO03/002170 or U.S. Pat. No. 5,720,720, 6,551,290 or 6,609,020. The entire disclosure of each of these Patent Applications and United States patents is hereby incorporated by reference into this specification. An even more preferred catheter and/or catheter system useful in the context of the present invention includes, but is not limited to, an intraparenchymal infusion cathetet and/or catheter system described in U.S. Pat. No. 6,093,180 (which is hereby incorporated by reference in its entirety). A most preferred catheter or catheter system useful in the context of the present invention is an intraparenchymal infusion catheter or catheter system described in International Patent Application Publication No. WO 03/077785 (which is hereby incorporated by reference in its entirety) and U.S. Pat. No. 6,609,020 (which is hereby incorporated by reference in its entirety).

The phrase “therapeutically effective dose” or “pharmaceutically effective dose”, which are used interchangeably herein, refers to that amount of GDNF sufficient to result in any amelioration, impediment, prevention or alteration of any biological symptom generally associated with a neurodegenerative disorder including, without limitation, PD by one skilled in the relevant art. In a preferred embodiment of the present invention, in conjunction with any of the above or below embodiments, GDNF is chronically infused directly into a target site at a dose of about 1 μg/day to about 100 μg/day. More preferably, GDNF is chronically infused directly into a target site at a dose of about 5 μg/day to about 50 μg/day. Even more preferably, GDNF is chronically infused directly into a target site at a dose of about 10 μg/day to about 75 μg/day. Even more preferably, GDNF is chronically infused directly into a target site at a dose of about 15 μg/day to about 50 μg/day. Even more preferably, r-metHuGDNF is chronically infused directly into a target site at a dose of about 20 μg/day to about 40 μg/day. Even more preferably, r-metHuGDNF is chronically infused directly into a target site at a dose of about 25 μg/day to about 30 μg/day. Even more preferably, r-metHuGDNF is chronically infused directly into a target site at a dose of about 15 μg/day to about 30 μg/day. Most preferably, r-metHuGDNF is chronically infused directly into a target site at a dose of about 25 μg/day to about 30 μg/day.

In the methods of the invention, GDNF is administered to more than one target site. Administration to the target sites may be simultaneous, sequential or separate. That is to say, GDNF may be administered to more than one target site at the same time, it may be administered to one target site and then immediately to another target site, or it may be administered to one target site and then another target site with a time delay between the two administrations.

Applicants also disclose herein the use of a pharmaceutically effective amount of GDNF including, but not limited to, r-metHuGDNF, and at least one pharmaceutically acceptable vehicle, excipient, or diluent in the preparation of a pharmaceutical composition for treating cognitive disorders or inhibiting cognitive decline associated with neurodegenerative disorders, including, without limitation, PD and dementia, wherein the composition is for administration to at least one of the SNc and a subthalamic region of the brain of a human in need thereof. In a preferred use of a pharmaceutically effective amount of GDNF and at least one pharmaceutically acceptable vehicle, excipient, or diluent in the preparation of a pharmaceutical composition for treating cognitive disorders or inhibiting cognitive decline associated with neurodegenerative disorders, including, without limitation, PD and dementia, the composition is for administration to at least one of the SNc, STN and ZI of the brain of a human in need thereof. In a more preferred use of a pharmaceutically effective amount of GDNF and at least one pharmaceutically acceptable vehicle, excipient, or diluent in the preparation of a pharmaceutical composition for treating cognitive disorders or inhibiting cognitive decline associated with neurodegenerative disorders, including, without limitation, PD and dementia, the composition is for administration to the SNc and/or ZI of the brain of a human in need thereof. In a more preferred use of a pharmaceutically effective amount of GDNF and at least one pharmaceutically acceptable vehicle, excipient, or diluent in the preparation of a pharmaceutical composition for treating cognitive disorders or inhibiting cognitive decline associated with neurodegenerative disorders, including, without limitation, PD and dementia, the composition is for administration to the SNc and/or the caudal ZI of the brain of a human in need thereof. In a more preferred use of a pharmaceutically effective amount of GDNF and at least one pharmaceutically acceptable vehicle, excipient, or diluent in the preparation of a pharmaceutical composition for treating cognitive disorders or inhibiting cognitive decline associated with neurodegenerative disorders, including, without limitation, PD and dementia, the composition is for administration to the SNc, the caudal ZI, and the posterodorsal putamen of the brain of a human in need thereof.

The inventive method has the effect, upon application to parkinsonian patients, of significantly reducing symptoms of Parkinson's disease, including tremor and dystonia. In addition, a clear improvement of disease-specific symptoms may result from use of the inventive methods disclosed herein insofar as motoricity, fine motoricity, and fine dexterity. In addition, mobility and concentration power may increase and reaction time may decrease. Pronunciation, facial expressiveness, posture, sense of smell, libido, sexual function, and emotional condition should be improved and state of mind should be brightened.

In yet another embodiment of the present invention, GDNF can be administered appropriately so that it can be used as a cognitive enhancer, to enhance learning, particularly as a result of dementias or trauma, or to inhibit cognitive decline and/or dementia, for example, in patients with PD. Alzheimer's disease, which has been identified by the National Institutes of Aging as accounting for more than 50% of dementia in the elderly, is also the fourth or fifth leading cause of death in Americans over 65 years of age. Four million Americans, 40% of Americans over age 85 (the fastest growing segment of the U.S. population), have Alzheimer's disease. Twenty-five percent of all patients with Parkinson's disease also suffer from Alzheimer's disease-like dementia. Previously, it has been shown that chronic intraputaminal administration of GDNF has application in treating or preventing cognitive disorders in humans. In particular, intraputaminal administration of GDNF has application in treating or preventing cognitive disorders and/or Alzheimer's disease-like dementia associated with PD.

### EXAMPLES

The following examples are provided for illustrative purposes only and are not to be construed as limiting the invention.

### Example 1

### Direct Infusion of GDNF into the Subthalamic Region and Substantia Nigra to Treat Parkinson's Disease

### Patient Inclusion Criteria

1. Diagnosis of Idiopathic Parkinson's Disease poorly controlled on optimum medication with significant functional disability.
2. Under the age of 65.
3. Appropriate surgical candidate with no significant co-morbid conditions.

### Patient Exclusion Criteria

1. Women of childbearing age.
2. Presence of clinically significant symptoms of dementia, depression, or significant memory impairment.

### Procedure

Subcortical and brainstem nuclei may be localised from high resolution MR images acquired under strict stereotactic conditions as previously reported (Patel, et al., 2002). Guide tubes may be used to ensure correct placement of one or more intraparenchymal brain catheters (0.45-1.25 mm) at the target site(s). Preferred guide tubes and catheters are described in U.S. Pat. No. 6,609,020 and International Patent Application Publication No. WO 03/077785, respectively (both of which are hereby incorporated by reference in their entireties). Surgery may be conducted under general anaesthesia. r-metHuGDNF primed SynchroMed pumps (Medtronic Inc, Minneapolis) may then be implanted in the upper abdominal region, subcutaneously or subfascially (beneath the anterior rectus sheath; subfascial placement may reduce the pump profile in the abdomen and improve cosmetic appearance). The catheters may be tunneled connecting the pumps to the indwelling intraparenchymal brain catheters.

r-metHuGDNF Infusion

r-metHuGDNF (Amgen Inc., Thousand Oaks, Calif.) is liquid formulated with 10 mM sodium citrate and 150 mM sodium chloride at a pH of 5.0. Following implantation, the SynchroMed pumps may be programmed to deliver a continuous infusion of r-metHuGDNF per target site per day. The pumps may be refilled as needed, e.g., monthly, with fresh solution. The infusion parameters may be altered to deliver drug at varying doses and/or rates in order to establish safe and clinically effective parameters, with repeat MRI monitoring at regular intervals. The vehicle solution to dilute the r-metHuGDNF may consist of sterile 10 mM citrate and 120 mM NaCl buffer at pH 5.5.

### Regimen of GDNF Infusion to the Target Site (Caudal Zona Incerta/SNc on Each Side)

1. Weeks 0 to 6—Buffer alone at 3 μl/hr.
2. Weeks 6 to 3 months—GDNF at a rate of 3 μl/hr (concentration of 100 μg/ml) therefore dose equivalent to 14.4 μg per STN/ZI per day.
3. 3 months to 12 months—GDNF at a rate of 3 μl/hr (concentration of 200 μg/ml therefore, equivalent to 28.8 μg per STN/ZI per day.
4. 12 months to 24 months—GDNF dose may be adjusted according to the clinical response.

### Clinical Assessments

1. UPDRS as per CAPIT criteria with video recording (at baseline and at intervals throughout treatment period).
2. Timed motor tests (at baseline and at intervals throughout treatment period).
3. Tremor Score (Fahn-Marsden Tolosa tremor rating score) (at baseline and at intervals throughout treatment period).
4. Patient Diaries (at baseline and at intervals throughout treatment period).
5. Neuropsychological tests (at baseline and at intervals throughout treatment period).
6. PDQ-39 and SF-(at baseline and at intervals throughout treatment period).
7. Blood Investigations (Full blood count, Renal, Liver Function test, Hypothalamic function tests (at baseline and intervals thereafter post surgery).
8. Vital signs (Pulse rate, blood pressure, respiratory rate, weight; (at baseline and intervals thereafter post surgery).

### Neuroimaging in Non Invasive, Relocatable Stereotactic Frame

1. MRI scans (T2 weighted and Diffusion Tensor Imaging)
   a) Baseline MR scan
   b) Preoperative plan scan
   c) Peroperative target confirmation scan
   d) 1 week post surgery
   e) 6 weeks post surgery
   f) 3, 6, 12, 18 and 24 months post surgery.
2. PET Scans (18-F Dopa and 11C-WAY 100635)
   a) Baseline
   b) 6, 12 and 24 months.

Direct infusion of GDNF into the lateral SNc and the caudal zonal incerta will improve motor symptoms in Parkinson's disease, including tremor and dystonia.

### Clinical Evaluation and Follow-Up

Clinical evaluations can be based on the Core Assessment Program for Intracerebral Transplantations (CAPIT) (Langston, J. W. et al., 1992), a validated protocol for evaluating surgical treatments of idiopathic PD. All patients can be evaluated on the Unified Parkinson's Disease Rating Scale (UPDRS) and undergo timed motor tests at baseline and at regular intervals following introduction of GDNF. Assessments may be performed in both off and on medication states. Preferably, before patients are assessed off medication, patients should fast and medications should be withdrawn overnight. Similar assessments may then be repeated after administration of L-dopa when the patients are “on”.

### Health-Related Quality of Life Outcome Measurement and Follow-Up

Patients may also be assessed using validated quality of life questionnaires: the 39-item Parkinson's Disease Questionnaire (PDQ 39) and the 36-item Medical Outcomes Study short form health survey (SF-36) may be used before surgery and at regular intervals after introduction of GDNF. Descriptive statistics (mean, standard deviation, range, 95% confidence interval) may be obtained for each variable. Comparisons over time may be made using Student's paired-samples t test.

### Neuropsychological Evaluation and Follow-Up.

Changes in medication (L-dopa equivalents) requirement may be measured and neuropsychology assessments with tests of verbal intellect, verbal and visual memory, attention, executive function, anxiety and depression as has been previously described (McCarter, R. J., et al., 2000) may be invoked as well. Preferably, any battery of cognitive tests used should be designed to minimize the possible confounding effects of both slowness of movement and movement difficulty on cognitive test results. Friedman's Related Samples test may be used to evaluate the significance of change over time in the rating scores. All analyses may be performed in SPSS. Patients may undergo pre-operative neuropsychological assessment and then be assessed at 12 and 24 months post implantation and GDNF exposure. The significance of changes in cognitive test performance may be evaluated using confidence intervals derived from the standard error of prediction (Lord and Novack, 1968; Atkinson, L., 1991) around the predicted true score at baseline. A significant change may be inferred if a score at either 12 or 24 months fall outside of the confidence interval of the baseline score. In addition, a PD control group consisting of patients who have undergone other forms of surgery for PD may be used to establish the effect of repeat cognitive assessment over a specified period. Of course, any control group should be comparable with the GDNF patient group in terms of years of education, age at surgery, duration of PD at surgery and NART estimated FSIQ (p>0.05).

### Scanning Procedures and Image Analysis

¹⁸F-dopa PET provides a measure of synaptic amino acid decarboxylase (AADC) activity and hence acts as an in vivo marker of dopamine storage and the functional integrity of dopamine terminals. Previous human and animal lesion studies have demonstrated that striatal ¹⁸F-dopa PET correlates with nigral cell numbers, dopamine content in striatal terminals (Garnett et al., 1983; Martin et al., 1989; Brooks et al., 1990(b); Pate et al., 1993) and the UPDRS off medication (Morrish, et al., 1998), in particular with the bradykinesia and rigidity sub scores (Otsuka, et al., 1996). Furthermore, it is possible to demonstrate progressive decline of striatal ¹⁸F-dopa uptake in patients with PD over time (Morrish, et al., 1998; Morrish, et al., 1996). ¹⁸F-dopa PET may be used here to assess striatal dopamine terminal function PD patients receiving chronic GDNF infusions.

The patients may undergo ¹⁸F-dopa PET pre-operatively, and at intervals postoperatively using an ECAT EXACT HR++ camera (CTI/Siemens 966; Knoxville, Tenn.) in 3D acquisition mode following withdrawal from medication for at least 12 hours. Patients may be dosed with 150 mg of carbidopa and 400 mg of entacapone; 1 hour later 111 MBq of ¹⁸F-dopa in normal saline may then be administered as an intravenous bolus at the start of scanning. The images can be acquired in 3D mode as 26 time frames over 94.5 minutes (1×30 seconds, 4×1 min, 3×2 min, 3×3 min and 15×5 mins). Parametric images of ¹⁸F-dopa influx constants (Ki) may be generated from time frames 25.5 to 94.5 minutes post injection using in house software (Brooks, D. J. et al., 1990; Rakshi, J. S. et al., 1999) based on the MTGA approach of Patlak and Blasberg (Patlak, C. S. & Blasberg, R. G., 1985)). Occipital counts from the same time frames were used to generate the tissue reference input function. Integrated images (time frames 25.5-94.5) may be used to identify the parameters required to transform the Ki images into standard stereotaxic MNI space. The transformation matrix may then be applied to the Ki images. After normalization a gaussian filter (6×6×6 mm) can be applied. Mean voxel values of the normalized Ki images may be compared throughout the midbrain and basal ganglia at baseline and intervals postoperatively using a paired Student's t-test in SPM99 after application of a mask to eliminate cortical signals and so reduce the number of statistical comparisons. Any regional increases in ¹⁸F-dopa uptake may be defined as a volume of interest and the mean Ki values for those volumes extracted using the appropriate SPM tool (Brett, M., et al., 2002).

The integrated images may be subsequently co-registered to each patient's MRI scan for region of interest (ROI) analysis. All MRIs may be reformatted in the AC-PC plane. The subsequent transformation matrix may then be applied to individual Ki images in order to transform them into the individual MRI space. Regions of interest (ROIs) may be traced on the MRI. For example, the head of the caudate and the dorsal putamen may be divided into anterior and posterior halves. The position of the catheter tip may be calculated relative to the AC-PC line and an oval region of interest (6 mm×12 mm) centered at the tip location in the axial plane. The ROI then may be copied onto 2 planes either side of the slice containing the calculated tip location, creating a 12 mm×6 mm×5 mm (0.36 cc) volume of interest centered on the catheter tip. The regions of interest can then be used to sample ¹⁸F activity on the parametric image.

### Example 2

### Direct Infusion of GDNF into the Subthalamic Region, Substantia Nigra, and Putamen to Treat Parkinson's Disease

PD patients with tremor predominant PD and/or worsening tremor and dystonia despite being treated with GDNF chronically infused intraputamenally may be treated by stereotactically placing one or more intraparenchymal infusion catheters into the caudal zona incerta, posterior to the subthalamic nucleus and about 2 mm above the substantia nigra compacta and continuing the infusion of GDNF intraputamenally. Guide tubes may be used to ensure correct placement of the intraparenchymal brain catheters (0.45-1.25 mm) at the target site(s). Preferred guide tubes and catheters are described in U.S. Pat. No. 6,609,020 and International Patent Application Publication No. WO 03/077785, respectively (both of which are hereby incorporated by reference in their entireties). The catheter will be connected to a Synchromed pump (Medtronic Inc. Minneapolis, Minn.) implanted in the anterior abdominal wall so that the patient will now have two pumps, both delivering GDNF to the brain. Intraputaminal infusion of GDNF may be continued at a rate of about 28.8 μg per putamen/day to about 45 ug per putamen/day while infusion to one or more STN/ZI sites will proceed according to the following regimen:
Weeks 0 to 6: Buffer alone at 3 μl/hr.
Weeks 6 to 18: GDNF at a rate of 3 μl/hr and concentration of 100 μg/ml (equivalent to 14.4 μg per STN/ZI per day).
Week 18 to 30: GDNF at a rate of 3 μl/hr and concentration of 200 μg/ml (equivalent to 28.8 μg per STN/ZI per day).

Pre-operative and post-operative monitoring and assessments of the patient may be conducted at appropriate intervals essentially as described above in Example 1.

### REFERENCES

Ai, Y., Markesbery, W., Zhang, Z., Grondin, R., Elseberry, D. D., Gerhardt, G. A., Gash, D. Intraputamenal infusion of GDNF in aged rhesus monkeys: distribution and dopaminergic effects. Journal of Comparative Neurology. 461: 250-261 (2003).
Arenas, E., Trupp, M., Akerud, P., Ibanez, C. F. GDNF prevents degeneration and promotes the phenotype of brain noradrenergic neurons in vivo. Neuron 15(6): 1465-73 (1995).
Atkinson, L. Three standard errors of measurement and the Wechsler Memory Scale—Revised. Psychological Assessment: A journal of consulting and clinical psychology, 3: 136-138 (1991).
Bhatia, K. P. & Marsden, C. D. The behavioural and motor consequences of focal lesions of the basal ganglia in man. Brain, 117 (Pt 4), 859-876 (1994).
Bankiewicz, K. S., Oldfield, E. H., Chiueh, C. C., Doppman, J. L., Jacobowitz, D. M., Kopin, I. J. Hemiparkinsonism in monkeys after unilateral internal carotid artery infusion of 1-methyl-4-phenyl-1,2,3,6-tetrahydropyridine. Life Sci., 39: 7-16 (1986).
Beck, K. D., Irwin, I, Valverde. J., Brennan, T. J., Langston, J. W., Hefti, F. GDNF induces a dystonia-like state in neonatal rats and stimulates dopamine and serotonin synthesis. Neuron, 16(3): 665-73 (1996).
Bensadoun, J. C., Deglon, N., Tseng, J. L., Ridet, J. L., Zumn, A. D., Aebischer, P. Lentiviral vectors as a gene delivery system in the mouse midbrain: Cellular and behavioral improvements in 6-OHDA model of Parkinson's disease using GDNF. Exp. Neurol., 164: 15-24 (2000).
Bjorklund, A., Kirik, D., Rosenblad, C., Georgievska, B., Lundberg, C., Mandel, R. J. Towards a neuroprotective gene therapy for Parkinson's disease: use of adenovirus, AAV and lentivirus vectors for gene transfer of GDNF to the nigrostriatal system in the rat Parkinson model. Brain Research, 886: 82-98 (2000a).
Bjorklund, A, Lindvall, A. Parkinson's disease gene therapy moves towards the clinic [Letter]. Nature Med, 6: 1207-1208 (2000b).
Bowenkamp, K. E., David, D., Lapchak, P. L., Henry, M. A., Grandholm, A. C., Hoffer B. J. 6-Hydroxydopamine induced the loss of the dopaminergic phenotype in substantia nigra neurons in the rat: a possible mechanism for restoration of the nigrostriatal circuit mediated by glial cell line-derived neurotrophic factor. Exp. Brain Res., 111: 1-7 (1996).
Brett, M., Anton, J., Valabregue, R., Poline, J. Region of interest analysis using an SPM toolbox. NeuroImage, 16 (2002).
Brooks, D. J. The relationship between locomotor disability, autonomic dysfunction, and the integrity of the striatal dopaminergic system in patients with multiple system atrophy, pure autonomic failure, and Parkinson's disease, studied with PET. Brain, 113 (Pt 5) 1539-1552 (1990a).
Brooks, D. J., Ibanez, V., Sawle, G. V., et al. Differing patterns of striatal 18F-dopa uptake in Parkinson's disease, multiple system atrophy, and progressive supranuclear palsy.[comment]. Annals of Neurology, 28: 547-555 (1990b).
Brown, P., Oliviero, A., Mazzone, P., Insola, A., Tonali, P., Di Lazzaro, V. Dopamine dependency of oscillations between subthalamic nucleus and pallidum in Parkinson's disease. J. Neurosci. 21(3): 1033-8 (2001).
Cass, W. A. GDNF selectively protects dopamine neurons over serotonin neurons against the neurotoxic effects of methamphetamine. J. Neurosci., 16: 8132-8139 (1996).
Choi-Lundberg, D. L., Lin, Q., Chang Y-N, Chiang, Y. L., Hay, C. M., Mohajeri, H. Dopaminergic neurons protected from degeneration by GDNF gene therapy. Science, 275: 838-841 (1997).
Connor, B., Kozlowski, D. A., Schaller, T., Tillerson, J. L., Davidson, B. L., Bohn, M. C. Differential effects of glial cell line-derived neurotrophic factor (GDNF) in the striatum and substantia nigra of the aged parkinsonian rat. Gene Therapy, 6: 1936-1951 (1999).
Costa, S., Mahmoud, M., Pearce, R. K. B., Jenner, P. Glial cell line-derived neurotrophic factor concentration dependently improves disability and motor activity in MPTP-treated common marmosets. European Journal Pharmacol., 412: 45-50 (2001).
Doder, R. E., Turjanski, N., Lees, A. J., Brooks, D. J. Tremor in Parkinson's disease and serotonergic dysfunction: an 11C-WAY 100635 PET study. Neurology. 25(60(4)):601-605 (2003).
Emborg-Knott, M. E., Domino, E. F. MPTP-induced hemiparkinsonism in nonhuman primates 6-8 years after a single unilateral intracarotid dose. Exp Neurol., 152: 214-220 (1998).
Francois, C., Yelnik, J., Tande, D., Agid, Y., Hirsch, E. C. Dopaminergic cell group A8 in the monkey: anatomical organization and projections to the striatum. J. Comp. Neurol. 414(3): 334-47 (1999).
Francois, C., Savy, C., Jan, C., Tande, D., Hirsch, E. C., Yelnik, J. Dopaminergic innervation of the subthalamic nucleus in the normal state, in MPTP-treated monkeys, and in Parkinson's disease patients. J. Comp. Neurol., 425(1): 121-9 (2000).
Freed, C. R., Greene, P. E., Breeze, R. E. Transplantation of embryonic dopamine neurons for severe Parkinson's disease. N. Engl. J. Med., 344: 710-719 (2001).
Garnett, E. S., Firnau, G., Nahmias, C. Dopamine visualized in the basal ganglia of living man. Nature, 305: 137-138 (1983).
Gash, D. M., Zhang, Z., Cass, W. A., Ovadia, L., Simmerman, L., Martin, D. Morphological and functional effects of intranigrally administered GDNF in normal rhesus monkeys. J. Comp. Neurol., 363: 345-358 (1995).
Gash, D. M., Zhang, Z., Ovadia, A., Cass, W. A., Yi, A., Simmerman, L. Functional recovery in parkinsonian monkeys treated with GDNF. Nature, 380: 252-255 (1996).
Gash, D. M., Zhang, Z., Gerhardt, G. A. Neuroprotective and neurorestorative properties of GDNF [Review]. Ann. Neurol., 44: S121-S125 (1998).
Gill, S. S., Patel, N. K., Hotton, G. R., O'Sullivan, K., McCarter, R., Bunnage, M., et al. Direct brain infusion of glial cell line-derived neurotrophic factor in Parkinson disease. Nat. Med., 9(5): 589-95 (2003).
Grondin, R., Zhang, Z., Elsberry, D. D., Gerhardt, G. A., Gash, D. M. Chronic intracerebral delivery of trophic factors via a programmable pump as a treatment for parkinsonism. In: Mouradian M M, editor. Parkinson's disease: Methods and Protocols. Totowa, N.J.: Humana Press, pp. 257-267 (2001).
Grondin, R., Zhang, Z., Ali, Y., Surgener, S. P., Loveland, A. D., Gerhardt, G. A., Gash, D. M. Chronic pulsatile delivery of GDNF into the substantia nigra increases motor speed and dopamine levels in the basal ganglia of MPTP-treated parkinsonian monkeys. Soc Neurosci. Abst., 28: 691-3 (2002a).
Grondin, R., Zhang, Z., Yi, A., et al. Chronic, controlled GDNF infusion promotes structural and functional recovery in advanced parkinsonian monkeys [comment]. Brain, 125: 2191-2201 (2002b).
Grondin, R., Zhang, Z., Ali, Y., Gash, D. M., Gerhardt, G. A. Intracranial delivery of proteins and peptides as a therapy for neurodegenerative diseases. Progress in Drug Research, 61:101-124 (2003).
Hagell, P. Dyskinesias following neural transplantation in Parkinson's disease. Nat. Neurosci., 5: 627-628 (2002).
Hassani, O. K., Francois, C., Yelnik, J., Feger, J. Evidence for a dopaminergic innervation of the subthalamic nucleus in the rat. Brain Res., 749(1): 88-94 (1997).
Herkenham, M., Little, M. D., Bankiewicz, K., Tang, S. C., Markey, S. P., Johannessen, J. N. Selective retention of MPP+ within the monoaminergic systems of the primate brain following MPTP administration: An in vivo autoradiographic study. Neuroscience, 40: 133-158 (1991).
Hou, J-G. G., Lin L-F. H., Mytilinenou, C., Glial cell line-derived neurotrophic factor exerts neurotrophic effects on dopaminergic neurons in vitro and promotes their survival and regrowht after damage by 1-methyl-4-phenylpyridinium. J. Neurochem., 66: 74-82 (1996).
Imbert, C., Bezard, E., Guitraud, S., Boraud, T., Gross, C. E. Comparison of eight clinical rating scales used for the assessment of MPTP-induced parkinsonism in the Macaque monkey. J. Neurosci. Meth, 96: 71-76 (2000).
Jellinger, K., Riederer, P., Tomonaga, M. Progressive supranuclear palsy: clinico-pathological and biochemical studies. J. Neural. Transm. Suppl., (16): 111-28 (1980).
Jellinger, K. Overview of morphological changes in Parkinson's disease. Adv. Neurol., 45 (1986).
Ka, J. Recent developments in the Pathology of Parkinson's disease. J. Neural Transm. 62: 347-376 (2002).
Kearns, C., Gash, D. M. GDNF protects nigral dopamine neurons against 6-hydroxydopamine in vivo. Brain Research, 672: 104-111 (1995).
Kirik, D., Rosenblad, C., Bjorklund, A., Mandel, R. J. Long-term rAAV-mediated gene transfer of GDNF in the rat Parkinson's model: Intrastriatal but not intranigral transduction promotes functional regeneration in the lesioned nigrostriatal system. J. Neurosci., 20: 4686-4700 (2000).
Kirik, D., Georgievska, B., Rosenblad, C., Bjorklund, A. Delayed infusion of GDNF promotes recovery of motor function in the partial lesion model of Parkinson's disease. Eur. J. Neurosci., 13: 1589-1599 (2001).
Kish, S. J., Shannak, K., Hornykiewicz, O. Uneven pattern of dopamine loss in the striatum of patients with idiopathic Parkinson's disease. Pathophysiology and clinical implications. N. Eng. J. Med, 318: 876-880 (1988).
Kolmac, C., Mitrofanis, J. Distribution of various neurochemicals within the zona incerta: an immunocytochemical and histochemical study. Anat. Embryol. (Berl) 199(3): 265-80 (1998).
Kolmac, C. I., Power, B. D., Mitrofanis, J. Patterns of connections between zona incerta and brainstem in rats. J. Comp. Neurol., 396(4): 544-55 (1998).
Kordower, J. H., Palfi, S., Chen, E-Y, Shuang. Y, Ma, Y., Sendera, T. Clinicopathological findings following intraventricular glial-derived neurotrophic factor treatment in a patient with Parkinson's disease. Ann. Neurol., 46: 419-424 (1999).
Kordower, J. H., Emborg, M. E., Bloch, J., Ma, S. Y., Chu, Y., Leventhal, L. Neurodegeneration prevented by lentiviral vector delivery of GDNF in primate models of Parkinson's disease. Science, 290: 767-773 (2000).
Kordower, Jeffrey, H., In vivo gene delivery of glial cell line-derived neurotrophic factor for Parkinson's disease. Ann. Neurol., 53(suppl 3): S120-S134 (2003).
Lang, A. E., Lozano, A. M. Parkinson's disease. First of two parts. [Review]. N. Engl. J. Med., 339: 1044-1053 (1998a).
Lang, A. E., Lozano, A. M. Parkinson's disease. Second of two parts. [Review]. N. Engl. J. Med., 339: 1130-1143 (1998b).
Langston, J. W. et al. Core assessment program for intracerebral transplantations (CAPIT). Mov. Disord., 7:2-13 (1992).
Lapchak, P. A., Araujo, D. M., Hilt, D. C., Jiao, S., Collins, F., Miyoshi, Y., et al. Topographical distribution of [¹²⁵I]-glial cell line-derived neurotrophic factor in unlesioned and MPTP-lesioned rhesus monkey brain following a bolus intraventricular injection. Brain Res., 789: 9-22 (1998).
Levy, R., Hutchison, W. D., Lozano, A. M., Dostrovsky, J. O. Synchronized neuronal discharge in the basal ganglia of parkinsonian patients is limited to oscillatory activity. J. Neurosci., 22(7): 2855-61 (2002).
Lindner, M. D., Winn, S. R., Baetge, E. E., Hammang, J. P., Gentile, F. T., Doherty, E., et al. Implantation of encapsulated catecholamine and GDNF-producing cells in rats with unilateral dopamine depletions and parkinsonian symptoms. Exp. Neurol., 132: 62-76 (1995).
Lin, C. S., Nicolelis, M. A., Schneider, J. S., Chapin, J. K. A major direct GABAergic pathway from zona incerta to neocortex. Science, 248(4962): 1553-6 (1990).
Lin, L-F. H., Doherty, D. H., Lile, J. D., Bektesh, S., Collins, F., GDNF: A glial cell line-derived neurotrophic factor for midbrain dopaminergic neurons. Science, 260: 1130-1132 (1993).
Lin, L-F. H., Zhang, T. J., Collins, F., Armes, L. G., Purification and initial characterization of rat B49 glial cell line-derived neurotrophic factor. J. Neurochem., 63: 758-768 (1994).
Lord, F. M., Novick, M. R. Statistical theories of mental test scores. Reading, Mass.: Addison-Wesley, 1968.
Ma, Y., Feigin, A., Dhawan, V., Fukuda, M., Shi, Q., Greene, P., Breeze, R., Fahn, S., Freed, C., Eidelberg, D. Dyskinesia after fetal cell transplantation for parkinsonism: a PET study (2002).
Magill, P. J., Bolam, J. P., Bevan, M. D. Dopamine regulates the impact of the cerebral cortex on the subthalamic nucleus-globus pallidus network. Neuroscience 106(2): 313-30 (2001).
Martin, W. R., Palmer, M. R., Patlak, C. S., Caine, D. B. Nigrostriatal function in humans studied with positron emission tomography. Annals of Neurology, 26:535-542 (1989).
Martin, D., Miller, G., Cullen, T., Fischer, N., Dix, D., Russell, D. Intranigral or intrastriatal injections of GDNF: effects on monoamine levels and behavior in rats. Eur. J. Pharmacol. 317(2-3): 247-56 (1996).
McCarter, R. J., Walton, N. H., Rowan, A. F., Gill, S. S., & Palomo, M. Cognitive functioning after subthalamic nucleotomy for refractory Parkinson's disease. J. Neurol. Neurosurg. Psychiatry, 69: 60-66 (2000).
Mitrofanis, J. and deFonseka, R. Organisation of connections between the zona incerta and the interposed nucleus. Anal. Embryol. (Berl), 204(2): 153-9 (2001).
Morrish, P. K., Sawle, G. V., Brooks, D. J. An [18F]dopa-PET and clinical study of the rate of progression in Parkinson's disease. Brain, 119 (Pt 2), 585-591 (1996).
Morrish, P. K., Rakshi, J. S., Bailey, D. L. Measuring the rate of progression and estimating the preclinical period of Parkinson's disease with [18F]dopa PET.[comment]. Journal of Neurology, Neurosurgery & Psychiatry, 64: 314-319 (1998).
Nutt, J. G., Burchiel, K. J., Comella, C. L., Jankovic, J., Lang, A. E., Laws, E. R., Jr., et al. Randomized, double-blind trial of glial cell line-derived neurotrophic factor (GDNF) in PD. Neurology, 60(1): 69-73 (2003).
Olson, L. Combating Parkinson's disease-step three. [Review]. Science, 290: 721-724 (2000).
Otsuka, M., Ichiya, Y., Kuwabara, Y., et al. Differences in the reduced 18F-Dopa uptakes of the caudate and the putamen in Parkinson's disease: correlations with the three main symptoms. Journal of the Neurological Sciences, 136: 169-173 (1996).
Ovadia, A., Zhang, Z., Gash, D. M. Increased susceptibility to MPTP toxicity in middle-aged rhesus monkeys. Neurobiol. Aging, 16: 931-937 (1995).
Pate, B. D., Kawamata, T., Yamada, T., et al. Correlation of striatal fluorodopa uptake in the MPTP monkey with dopaminergic indices. Annals of Neurology, 34: 331-338 (1993).
Patlak, C. S. & Blasberg, R. G. Graphical evaluation of blood-to-brain transfer constants from multiple-time uptake data. Generalizations. J. Cereb. Blood Flow Metab. 5: 584-590 (1985).
Pahwa, R., Wilkinson, S., Smith, D., et al. High-frequency stimulation of the globus pallidus for the treatment of Parkinson's disease. Neurology, 49: 249-253 (1997).
Patel, N. K., Heywood, P., O'Sullivan, K., Love, S., Gill, S. S. MRI-directed subthalamic nucleus surgery for Parkinson's disease. Stereotact. Funct. Neurosurg., 78(3-4): 132-45 (2002).
Paulus, W. J. K. The neuropathologic basis of different clinical subgroups of Parkinson's disease. J. Neuropathol. Exp. Neurol. November.; 50(6):743-55 (1991).
Perier, C., Vila, M., Feger, J., Agid, Y., Hirsch, E. C. Functional activity of zona incerta neurons is altered after nigrostriatal denervation in hemiparkinsonian rats. Exp. Neurol. 162(1): 215-24 (2000).
Power, B. D. and Mitrofanis, J. Ultrastructure of afferents from the zona incerta to the posterior and parafascicular thalamic nuclei of rats. J. Comp. Neurol. 451(1): 33-44 (2002).
Rakshi, J. S., et al. Frontal, midbrain and striatal dopaminergic function in early and advanced Parkinson's disease A 3D [¹⁸F]-dopa PET study. Brain, 122 (Pt 9): 1637-1650 (1999).
Roger, M. and Cadusseau, J. Afferents to the zona incerta in the rat: a combined retrograde and anterograde study. J. Com.p Neurol. 241(4): 480-92 (1985).
Rosenblad, C., Martinez-Serrano, A., Bjorklund, A. Intrastriatal glial cell line-derived neurotrophic factor promotes sprouting of spared nigrostriatal dopaminergic afferents and induces recovery of function in a rat model of Parkinson's disease. Neuroscience, 82: 129-137 (1998).
Smith, R. D., Zhang, Z., Kurlan, R., McDermott, M., Gash, D. M. Developing a stable bilateral model of parkinsonism in rhesus monkeys. Neuroscience, 52: 7-16 (1993).
Smith, Y., Kieval, J. Z. Anatomy of the dopamine system in the basal ganglia. [Review]. Trends Neurosci., 23(10 Suppl): S28-S33 (2000).
The Deep-Brain Stimulation for Parkinson's Disease Study Group. Deep brain stimulation of the subthalamic nucleus or the pars interna of the globus palidus in Parkinson's Disease. New England Journal Medicine, 345: 956-963 (2001).
Tomac, A., Lindqvist, E., Lin, L-F. H., Ogren, S. O., Young, D., Hoffer, B. J., et al. Protection and repair of the nigrostriatal dopaminergic system by GDNF in vivo. Nature, 373: 335-339 (1995a).
Tomac, A., Widenfalk, Lin, L-F. H., Kohno T., Ebendal, T., Hoffer, B. J., et al. Retrograde axonal transport of glial cell line-derived neurotrophic factor in the adult nigrostriatal system suggests a trophic role in the adult. Proc. Natl. Acad. Sci. USA, 92: 8274-8278 (1995b).
Zhang, Z., Miyoshi, Y., Lapchak, P. A., Collins, F., Hilt, D., Lebel, C., et al. Dose response to intraventricular glial cell line-derived neurotrophic factor administration in parkinsonian monkeys. J. Pharmacol. Exp. Ther., 282: 1396-1401 (1997).

1. A method of treating Parkinson's disease in a human that comprises administering a pharmaceutical composition comprising a pharmaceutically effective amount of a GDNF protein product and at least one pharmaceutically acceptable vehicle, excipient, or diluent to a target site within one or both putamen and at least one other target site selected from the group consisting of the substantia nigra compacta, the subthalamic nucleus and the zona incerta of a human in need thereof.

2. A method of treating Parkinson's disease in a human that comprises assessing dopaminergic function in one or both putamen of said human, pre-operatively;
identifying at least one target site of dopaminergic dysfunction within one or both putamen;
administering a pharmaceutical composition comprising a pharmaceutically effective amount of a GDNF protein product and at least one pharmaceutically acceptable vehicle, excipient, or diluent to one or more of said target sites within the putamen and at least one other target site selected from the group consisting of the substantia nigra compacta, the subthalamic nucleus and the zona incerta;
assessing dopaminergic function at one or more of said putamen target sites post-operatively, at least once; and, optionally
assessing serotonergic function at one or more of said other target sites, at least once.

3. A method of increasing the function of dopaminergic neurons in a human that comprises administering a pharmaceutical composition comprising a pharmaceutically effective amount of a GDNF protein product and at least one pharmaceutically acceptable vehicle, excipient, or diluent to a target site within one or both putamen and at least one other target site selected from the group consisting of the substantia nigra compacta, the subthalamic nucleus and the zona incerta of a human in need thereof.

4. A method of increasing the uptake of dopamine by dopaminergic neurons in a human that comprises administering a pharmaceutical composition comprising a pharmaceutically effective amount of a GDNF protein product and at least one pharmaceutically acceptable vehicle, excipient, or diluent to a target site within one or both putamen and at least one other target site selected from the group consisting of the substantia nigra compacta, the subthalamic nucleus and the zona incerta of a human in need thereof.

5. A method of regenerating dopaminergic neurons in a human that comprises administering a pharmaceutical composition comprising a pharmaceutically effective amount of a GDNF protein product and at least one pharmaceutically acceptable vehicle, excipient, or diluent to a target site within one or both putamen and at least one other target site selected from the group consisting of the substantia nigra compacta, the subthalamic nucleus and the zona incerta of a human in need thereof.

6. A method of protecting dopaminergic neurons susceptible to degeneration in a human that comprises administering a pharmaceutical composition comprising a pharmaceutically effective amount of a GDNF protein product and at least one pharmaceutically acceptable vehicle, excipient, or diluent to a target site within one or both putamen and at least one other target site selected from the group consisting of the substantia nigra compacta, the subthalamic nucleus and the zona incerta of a human in need thereof.

7. The method of claim 1 wherein said target site within one or both putamen is the central region of said putamen.

8. The method of claim 1 wherein said target site within one or both putamen is the postero-dorsal region of said putamen.

9. The method of claim 1, wherein said vehicle, excipient, or diluent comprises sodium chloride and sodium citrate.

10. The method of claim 1, wherein said GDNF protein product is r-metHuGDNF.

11. The method of claim 2, wherein assessing dopaminergic function comprises assessing dopamine uptake or dopamine storage.

12. The method claim 1 wherein said at least one other target site is selected from the group consisting of the substantia nigra compacta, the subthalamic nucleus and the caudal zona incerta.

13. The method of claim 1 wherein said at least one other target site is the caudal zona incerta, posterior to the subthalamic nucleus and about 2 mm above the substantia nigra compacta.

14. A method of treating Parkinson's disease in a human that comprises administering a pharmaceutical composition comprising a pharmaceutically effective amount of a GDNF protein product and at least one pharmaceutically acceptable vehicle, excipient, or diluent to at least one target site selected from the group consisting of the substantia nigra compacta, the subthalamic nucleus and the zona incerta of a human in need thereof.

15. A method of treating Parkinson's disease in a human that comprises assessing dopaminergic function in one or more regions of the brain of said human selected from the group consisting of the substantia nigra compacta, the subthalamic nucleus and the zona incerta, pre-operatively;
identifying at least one target site of dopaminergic dysfunction within one or more of said regions of the brain;
administering a pharmaceutical composition comprising a pharmaceutically effective amount of a GDNF protein product and at least one pharmaceutically acceptable vehicle, excipient, or diluent to at least one of said target sites;
assessing dopaminergic function at one or more of said target sites post-operatively, at least once; and, optionally
assessing serotonergic function within at one least one brain region of said brain, at least once.

16. A method of increasing the function of dopaminergic neurons in a human that comprises administering a pharmaceutical composition comprising a pharmaceutically effective amount of a GDNF protein product and at least one pharmaceutically acceptable vehicle, excipient, or diluent to at least one target site selected from the group consisting of the substantia nigra compacta, the subthalamic nucleus and the zona incerta of the brain of said human.

17. A method of increasing the uptake of dopamine by dopaminergic neurons in a human that comprises administering a pharmaceutical composition comprising a pharmaceutically effective amount of a GDNF protein product and at least one pharmaceutically acceptable vehicle, excipient, or diluent to at least one target site selected from the group consisting of the substantia nigra compacta, the subthalamic nucleus and the zona incerta of the brain of said human.

18. A method of regenerating dopaminergic neurons in a human that comprises administering a pharmaceutical composition comprising a pharmaceutically effective amount of a GDNF protein product and at least one pharmaceutically acceptable vehicle, excipient, or diluent to at least one target site selected from the group consisting of the substantia nigra compacta, the subthalamic nucleus and the zona incerta of the brain of said human.

19. A method of protecting dopaminergic neurons susceptible to degeneration in a human that comprises administering a pharmaceutical composition comprising a pharmaceutically effective amount of a GDNF protein product and at least one pharmaceutically acceptable vehicle, excipient, or diluent to at least one target site selected from the group consisting of the substantia nigra compacta, the subthalamic nucleus and the zona incerta of the brain of said human.

20. The method of claim 14, wherein said vehicle, excipient, or diluent comprises sodium chloride and sodium citrate.

21. The method of claim 14, wherein said GDNF protein product is r-metHuGDNF.

22. The method of claim 15, wherein assessing dopaminergic function comprises assessing dopamine uptake or dopamine storage.

23. The method of claim 14, wherein said target site is selected from the group consisting of the substantia nigra compacta, the subthalamic nucleus and the caudal zona incerta.

24. The method of claim 14, wherein said a target site is the caudal zona incerta, posterior to the subthalamic nucleus and about 2 mm above the substantia nigra compacta.

25-60. (canceled)
